(19) United States
(12) Patent Application Publication
(10) Pub. No.: US 2025/0346883 A1
(43) Pub. Date: Nov. 13, 2025

(54) DENSITY GRADIENT CHEMOSTAT FOR ADAPTED LABORATORY EVOLUTION

(71) Applicant: THE REGENTS OF THE UNIVERSITY OF CALIFORNIA, Oakland, CA (US)

(72) Inventors: Jeff Shovlowsky Bowman, San Diego, CA (US); Maile Heyer, La Jolla, CA (US); Benjamin Klempay, La Jolla, CA (US); Luke Fisher, La Jolla, CA (US); Joseph Riley, La Jolla, CA (US)

(21) Appl. No.: 18/867,424

(22) PCT Filed: May 22, 2023
(86) PCT No.: PCT/US2023/023142

§ 371 Date: Nov. 19, 2024

Related U.S. Application Data
(60) Provisional application No. 63/344,498, filed on May 20, 2022.

(51) Int. Cl. C12N 15/01; C12M 1/00; C12M 1/26; C12N 1/20

(52) U.S. Cl. CPC C12N 15/01; C12M 23/34; C12M 29/06; C12M 33/04; C12N 1/20

(57) ABSTRACT

A system and method for adaptive laboratory evolution (ALE) employs density stratified layers of cell growth media within a chemostat to form an interface between the layers, creating a gradient with an increasing concentration of a stressor and nutrients. Cells are encouraged to evolve by providing greater nutrients at higher concentrations of the stressor. The chemostat includes ports for accessing the media and cells at different layers for adaptation analysis.

### RELATED APPLICATIONS

This application claims the benefit of the priority of U.S. Provisional Application No. 63/344,498, filed May 20, 2022, which is incorporated herein by reference in its entirety.

### FIELD OF THE INVENTION

The present invention relates to a system and method for culturing microbial strains and more specifically to a system and method that support adaptive evolution of microbial strains.

### BACKGROUND

Adaptive Laboratory Evolution (“ALE”) is an experimental method that uses a specific set of conditions to accelerate Darwinian-style evolution to produce microbial strains with a desired phenotype. The experimental conditions determine the fitness levels of phenotypes present in the population, increasing the relative fitness of the desired phenotype. The resulting natural selection chooses mutants with the desired phenotype, directing the evolutionary trajectory of the microbial population. Sequencing of ALE-evolved strains reveals the genetic mutations responsible for these observed phenotypes. These genetic changes are often the result of a complex network of changes that go beyond the current understanding of gene expression and cellular machinery. Thus, ALE can be a powerful tool for strain development because the selective pressure drives cells to optimize their cellular machinery, without a priori knowledge on the matter. The self-optimized strain can then be further manipulated through genome engineering. During ALE, the laboratory environment is precisely controlled, allowing the experimental conditions to be linked to the observed phenotypes for replicable evidence on how factors influence evolutionary outcomes. Furthermore, samples from multiple timepoints during the experiment can be stored indefinitely and revived for a genetic and phenotypic record of the experiment. These records can show the evolutionary trajectory that took place to achieve the final strain, enhancing understanding of the molecular basis of evolutionary adaptation and population dynamics.

A common use of ALE is to study microbial stress responses to growth-limiting conditions and adaptations that provide increased tolerance. In both natural and unnatural environments, microorganisms are exposed to stressors that impact their ability to function, limit their growth, and affect the broader microbial community and ecosystem. Understanding microbial stress, stress tolerance, and evolution towards stress tolerance is the focus of many questions surrounding microbial systems such as industrial biotechnology, clinical infections and medicines, soil health, sanitization, astrobiology, antibiotic resistance, and much more. For example, high soil salinity caused by drought, exposure to ethanol during industrial bioproduction, and acidic conditions in the human intestinal tract are stressors that have driven genetic adaptations for increased tolerance in microbial populations, whether naturally or during experiments.

Despite the use of ALE to study microbial stress responses, there are critical challenges to its execution: the wild type must be able to survive the initial conditions, the selective pressure must sort out strains with increased stress tolerance, and stress tolerance must be coupled with increased fitness for natural selection to act on the favored phenotype, all while maintaining a controlled environment. If these challenges are successfully addressed, the ALE experiment should produce microbial strains that have specifically evolved in response to the experimental conditions.

The first challenge for laboratory evolution towards stress tolerance is that the experiment must expose wild type cells to stress, potentially harming the cells beyond what is required for successful evolution. A “stressor” can be defined as an external molecule, such as an antibiotic, or a physical property, such as high temperature or pressure, which causes an environment to approach or surpass the limit of an organism's physiological tolerance and impairs its ability to function. While stress may not always be fatal to microorganisms, it can cause irreversible damage, prevent growth and reproduction, and trigger a wide range of regulatory changes that alter gene expression, or even alter the genome's structure itself such as by inducing mutagenesis. Some of these changes may fulfill the goals of ALE to produce strains with better fitness against a stressor, however excessive damage can result in experiment failure.

In contrast to the first challenge, the second challenge requires sufficient external stress to select for mutants with stress tolerance and against other phenotypes. If the stress level is too weak, there will not be enough selective pressure on cells to maintain energy-costly stress adaptations, or the low levels of selection may not select for the mutations with the highest tolerance to stress. Thus, an effective ALE experiment must apply the appropriate degree of evolutionary pressure (stress) to sort out mutants with the desired phenotype (stress tolerance).

Achieving an appropriate balance for stress levels to select for adapted strains is a challenge due to an incomplete understanding of how microbes respond to stress, and how to measure that stress. The inability to measure microbial stress limits common ALE experimental methods in which the level of stress is increased artificially. Common ALE experiments are performed with batch cultures or a continuous culture device, known as a chemostat. During batch culture experiments, cell populations are transferred at fixed intervals from flask to flask with increasing amounts of selective pressure. Chemostats, on the other hand, operate in a single bioreactor, maintaining a continuous culture of cells steady-state growth by continuously supplying media and removing waste. The fresh supply of media lacks one key nutrient so that cells are starved and grow at a reduced rate. The selective pressure is increased over time by changing the components of the inflowing media. Both methods increase the level of stress and selection by a predetermined amount at a predetermined time, imposing a time constraint on evolution, without an understanding of how the cells have responded or if they will be able to respond. These methods, therefore, have a limited ability to achieve a balance between too little and too much stress.

The third challenge addresses the fundament of Darwinian evolution that ALE is based on. Specifically, during ALE, the favorable phenotype must correspond with increased fitness because natural selection will choose mutants based on their fitness in the environment. Typically, increased fitness of bacterial cells is determined by their ability to survive and replicate at a faster rate in less favorable conditions, however, other components of fitness besides growth rate exist as well. For ALE experiments that seek to increase tolerance to a stressor, the favorable phenotype, stress tolerance, is directly linked to increase fitness because these strains survive better. The difficulty, however, is controlling the conditions such that adapting stress-tolerance has more advantages than the energetic cost of these adaptations and is more fit than other phenotypes, such as those that allow evasion of the stress altogether. If all cells are exposed to a stressful agent, then adapting tolerance is favorable. The challenge, then, lies in determining the optimal degree of stress to effectively implement Nietzsche's famed aphorism: “what doesn't kill [the cells] makes them stronger.”

A spatial increase of stress allows cells to evolve at their own pace, removing any time constraints on evolution. The wild type can survive in low-stress regions while stress-tolerant mutants are sorted out simply by their existence in high-stress regions, addressing the first two challenges of ALE. For the third challenge to ALE, the relative fitness of stress-tolerant phenotypes is increased because mutants are provided with unused space and nutrients. Mutations for stress-tolerance, therefore, will be naturally selected for fixation in the population. Microbial populations can be further pushed to evolve to the stressor by increasing the nutrient concentration in high-stress regions, making colonization of the stressful regions the better evolutionary strategy. Lastly, an added benefit of using a structured gradient is that the heterogeneous environment supports diversity in the population, facilitating adaptation.

A spatial increase in stress facilitates adaptation by providing intermediate steps and accelerates the rate of evolution by maintaining the diversity present in the population. ALE experiments with the MEGA-plate and microfluidic gradient chamber, by Baym et al. (Spatiotemporal microbial evolution on antibiotic landscapes. Science 353, 1147-1151 (2016)) and Zhang et al. (Acceleration of Emergence of Bacterial Antibiotic Resistance in Connected Microenvironments. Science 333, 1764-1767 (2011)) respectively, used a spatial increase in antibiotic to produce cells with high antibiotic resistance. By providing intermediate steps with moderate amounts of selective pressure, both experiments produced strains with high levels of microbial resistance. In Zhang et al.'s work, when microorganisms were exposed to a high level of antibiotics without intermediate steps, they were unable to adapt. The gradual increase in stress, therefore, allowed cells to reach a higher level of antibiotic resistance. Additionally, the heterogeneous environment, as opposed to a homogeneous one, increased the rate of adaptation. The connected microenvironments provide ecological opportunity for populations to diversify and potentially evolve new mutants with the ability to cope with higher levels of stress. Earlier work showed that a structured, heterogeneous environment increased the diversity in a population by allowing bacteria to occupy different niches, reducing competition. Homogeneous environments, like those in chemostats and shaken batch cultures, on the other hand, have reduced genetic diversity in which populations may adopt a “quick fix” mutation. Furthermore, studying evolution within a heterogeneous environment has better applications to evolution outside of the laboratory. For example, pathogenic bacteria colonize on heterogeneous surfaces that allow for higher diversity, potentially increasing the rate of antibiotic-resistant phenotypes compared to in vitro studies with homogeneous cultures.

Despite the evidence presented in favor of evolution studies in a heterogeneous environment, ALE experiments are still dominated by use of batch cultures and chemostats with few examples of ALE using a heterogeneous set up. Furthermore, the available methods, such as the MEGA-plate and microfluidic gradient chamber, are currently limited to studying antibiotic resistance with model organisms, excluding other organisms with great potential for biotechnological use. For example, marine microorganisms with stress tolerance adaptations, such as desiccation resistance and high salinity tolerance.

Accordingly, the need remains for an approach, and a bioreactor, which supports adaptive evolution of microbial strains.

### SUMMARY

The inventive scheme enables the adaptive evolution of bacterial strains to conditions that are too challenging for traditional adaptive evolution culturing techniques. This novel approach to ALE employs a density gradient in a chemostat to physically partition the bacterial strain for the conditions. The multi-layer bioreactor, referred to herein as the “Multilayered Instrument for Continuous Adaptive Laboratory Evolution” or “MICALE,” has two distinct media types separated by density. The less-dense top layer media (“TLM”) operates as a traditional chemostat and is permissive for steady-state growth of the wild type. The denser bottom layer media (“BLM”) has an added stressor, such as a salt or antibiotic, but has no in-flowing or out-flowing media, allowing for batch-culture-style growth of an evolved strain. The media types mix at their interface, creating a spatial gradient from “mostly TLM” to “mostly BLM,” thereby forming an increasing gradient in the concentration of a stressor and nutrients. Cells with stress tolerant phenotypes can colonize the lower regions of media at their own pace, with no time constraints imposed on evolution.

If cells successfully adapt to the stressful conditions, they will be rewarded with more nutrients and uncontrolled growth. The nutrient difference, along with the controlled growth in the top permissive layer, acts as a sufficient resource limitation to encourage adaptation to conditions in the lower layer. Using valves placed vertically along the side of the chemostat, sampling can occur along the entire stress gradient. In the exemplary embodiment, these sampling areas are divided into four sections: the permissive layer (PL) in which media is constantly flowing and is majority TLM, the interface (IL) that sits at the now-blurred boundary between the two media types, and the stress layer (SL) followed by the base (BL) that contain majority BLM and remain static.

To evaluate the inventive approach, with high concentrations of sodium chloride (NaCl,) magnesium chloride (MgCl2,) and the antibiotic ciprofloxacin in the BLM, Escherichia coli MG1655 (E. coli) was grown in the MICALE bioreactor for two to four weeks. Each selected stressor exposed the bacteria populations to a different type of hostility intended to drive the course of genetic adaptation. High salinity adds osmotic and ionic stress to cells and may reduce the solubility of metabolites. Chaotropic stress, caused by solutes such as MgCl₂, disrupts intermolecular forces thereby destabilizing, denaturing, and inhibiting important molecules in a cell such as proteins, enzymes, and membranes. Ciprofloxacin (“Cipro”), a quinolone antibiotic, targets enzymes essential for bacterial DNA replication and is commonly used to treat infections caused by E. coli and other Gram-negative bacteria. It may be noted that there is some disagreement as to whether the toxic effect of antibiotics is a type of cellular stress; antibiotics have specific target sites and modes of action, compared to stressors like magnesium chloride that act on multiple cellular mechanisms. Nonetheless, it has been shown experimentally that ciprofloxacin and other antibiotics trigger stress-induced mutagenesis, a bacterial response to stress characterized by a transient mutator state. Therefore, stressing E. coli ciprofloxacin, in addition to NaCl and MgCl₂, allows evaluation of differing evolutionary responses to three unique types of stress.

In one aspect of the invention, a system for adaptive laboratory evolution of microorganisms includes a chemostat comprising a chamber, a fresh media inlet, and a waste media outlet, the chamber configured to retain a growth medium comprising density stratified layers, wherein an interface between the layers creates a gradient with an increasing concentration of a stressor and a nutrient progressing toward a bottom of the chamber, wherein an absence of physical barriers within the chamber permits the microorganisms to move freely within the growth medium; and a plurality of access ports through the chamber, each access port disposed at a different height of the chemostat to provide access for extracting the growth medium at different vertical levels of the chamber. The density stratified layers include a top layer media (TLM) and a bottom layer media (BLM) each comprising the growth medium, where the TLM further includes a dilutant added to the growth medium and where the nutrient within the growth medium increases a density of the BLM relative to the TLM. In some embodiments, the growth medium is Luria broth, and wherein, in the TLM, the Luria broth is diluted to 50%. The nutrient may be a carbohydrate, which may be one or more sugar selected from the group consisting of sucrose, glucose, maltose, lactose, and trehalose.

In some embodiments, the microorganisms are Escherichia coli and the stressor compound may be one or more of a saline compound, a chaotropic compound, and an antibiotic.

Each of the access ports is configured for extracting the growth medium and associated micro-organisms located at different vertical levels within the chamber corresponding to a different layer within the growth medium. The different layers may include a permissive layer (PL) configured for wild-type growth, an interface layer (IL) disposed below the PL, a stress layer (SL) below the IL, and a base layer (BL) disposed at the lower portion of the chamber. The fresh media inlet and the waste media outlet may be configured to maintain a constant flow of the growth medium into and out of the chamber at a height of the chamber corresponding to the PL.

In another aspect of the invention, a method for adaptive laboratory evolution of microorganisms includes: introducing microorganisms into a chemostat containing a growth medium comprising density stratified layers, wherein an interface between the layers creates a gradient with an increasing concentration of a stressor and a nutrient progressing toward a bottom of the chemostat, wherein an absence of physical barriers within the chemostat permits the microorganisms to move freely within the growth medium; and extracting the growth medium via access ports disposed at different vertical levels of the chemostat. The density stratified layers include a top layer media (TLM) and a bottom layer media (BLM) each comprising the growth medium, where the TLM further includes a dilutant added to the growth medium and where the nutrient within the growth medium increases a density of the BLM relative to the TLM. In some embodiments, the growth medium is Luria broth, and wherein, in the TLM, the Luria broth is diluted to 50%.

### BRIEF DESCRIPTION OF THE DRAWINGS

FIG. 1 is a diagram of the inventive multi-layer chemostat according to an embodiment of the invention.

FIG. 2 is a flow diagram of the steps used in testing for evolutionary responses in samples processed using the inventive chemostat.

FIGS. 3A-3C illustrate results of adaptation analysis of samples processed using the inventive MICALE chemostat, where FIG. 3A plots optical density (OD₆₀₀) of the permissive, interface, and stress layers samples compared to ancestor strains; FIG. 3B is a sample plot showing exponential growth rate u and cell proliferation based on the change in OD (max-min); FIG. 3C provides a sample dose-response curve, which represents a sample's relative response to increasing doses of a stressor. The value for ED50 is the effective dose of the stressor at which a 50% decrease in the maximal response (100%) is observed.

FIG. 4 is a sample plot of exponential growth rate obtained by rolling regression based on OD₆₀₀ data.

FIGS. 5A-5B are dose response curves for relative exponential growth rate over an increasing concentration of antibiotic (cipro) in units of minimum inhibitory concentration (MIC) for two samples taken from the interface layer (IL).

FIG. 6A plots refractometer salinity measurements of the permissive and interface layers during a high salinity stress run; FIGS. 6B and 6C plot density measurements for each of the four layers (PL, IL, SL, BL) during chaotropic and antibiotic stress runs, respectively.

FIGS. 7A-7B provide results for high salinity stress runs, where FIG. 7A plots optical density measurements for each of the four layers, and FIG. 7B plots media flow rates with PL OD₆₀₀.

FIGS. 8A-8B provide results for chaotropic stress runs, where FIG. 8A plots optical density measurements for each of the four layers, and FIG. 8B plots media flow rates with PL OD₆₀₀.

FIGS. 9A-9C provide results for high salinity stress runs, where FIG. 9A plots optical density measurements for each of the four layers; FIG. 9B plots optical density measurements without the permissive layer (PL); and FIG. 9C plots media flow rates with PL OD₆₀₀.

FIG. 10 provides high salinity adaptation analysis OD₆₀₀ curves for samples of increasing NaCl concentrations taken from different layers of the MICALE chemostat.

FIG. 11 provides chaotropic adaptation analysis OD₆₀₀ curves for samples of increasing MgCl₂ concentrations taken from different layers of the MICALE chemostat.

FIGS. 12A-12B provide antibiotic adaptation analysis OD₆₀₀ curves for samples of increasing cipro concentrations taken from different layers of the MICALE chemostat, over two trials.

### DETAILED DESCRIPTION OF EMBODIMENTS

### Definitions

Unless otherwise defined, the technical and scientific terms used herein have the same meaning as commonly understood by one of ordinary skill in the art. Expansion and clarification of some terms are provided herein. All publications, patent applications, patents and other references mentioned herein, if not otherwise indicated, are explicitly incorporated by reference.

As used herein, the singular forms “a,” “an”, and “the” include plural referents unless the context clearly dictates otherwise.

The term “biological cell” or “cell” refers to any cell from an organism, including, but not limited to, insect, microbial, fungal (for example, yeast), algal, or animal, (for example, mammalian) cells.

The terms “area” and “layer” refer to a volume of media within the chemostat that is distinguished from other areas or layers by density stratification rather separation by a physical, e.g., mechanical, barrier.

As used herein, “broth” and “medium” refer to a fluid composition suitable for cultivation of micro-organisms within a chemostat. A cultivation medium is selected based on the elemental composition and the biosynthetic capacity of a given microorganism. While the illustrative examples described herein utilize the medium of Luria Broth for cultivation of E. coli, those of skill in the art will recognize that selection of an appropriate medium will depend on the organism to be cultured. As such, the terms “broth”, media,” and medium” are not intended to be limited to the examples specifically presented herein.

Several acronyms are used throughout the written description. Table 1 provides a reference list of the acronyms and their meanings.

TABLE 1

| ACRONYM | MEANING |
|---|---|
| MICALE | Multilayered Instrument for Continuous |
| ALE | Adaptive Laboratory Evolution |
| TLM | Top Layer Media |
| BLM | Bottom Layer Media |
| PL | Permissive Layer |
| IL | Interface region |
| SL | Stress Layer |
| BL | Base region |
| OD₆₀₀ | Optical Density (at λ = 600 nm) |
| MIC | Minimum Inhibitory Concentration |
| WGS | Whole Genome Sequencing |
| EPS | Extracellular polysaccharides |

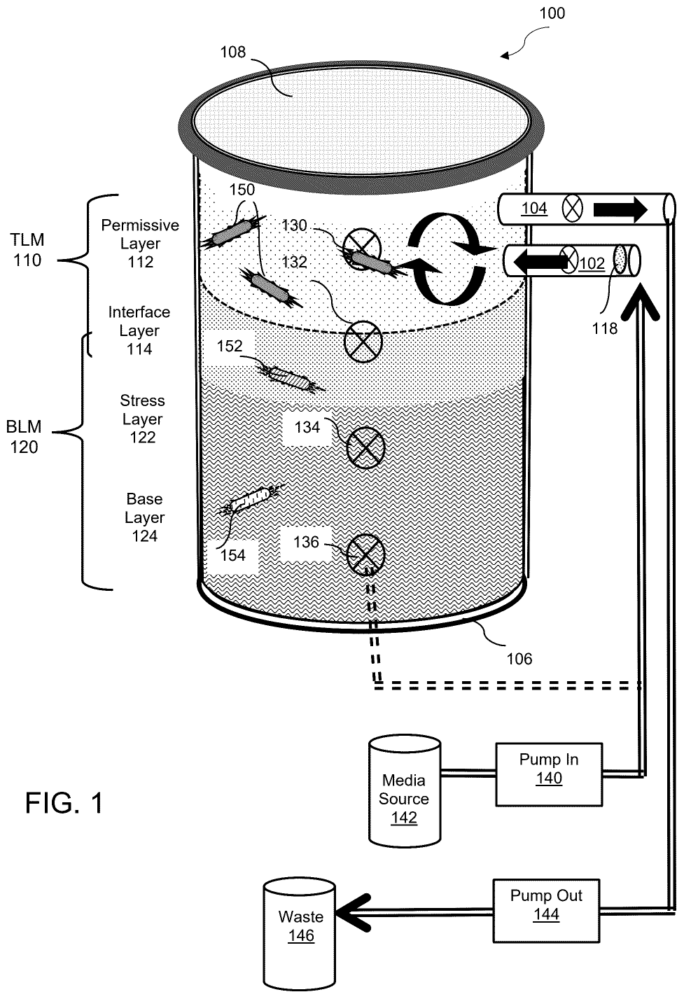

FIG. 1 diagrammatically illustrates the multi-layer configuration of the inventive chemostat, also known as “MICALE”. The chemostat 100 contained two media layers. The Top Layer Media (TLM) 110, forming the top ˜25% of the total media volume, was Luria Broth (LB) diluted with filtered water (MilliQ water) to about 40-60%, and more preferably about 50%. The Bottom Layer Media (BLM) 120, making up the lower ˜75% of the volume, was fully concentrated LB. In the test set-up, the total media volume was about 1,150 mL, with ˜250 mL of TLM and ˜900 mL of BLM. The density of the BLM was increased by adding a densifying nutrient, e.g., a carbohydrate. In the present implementation, 100 g/L of sucrose, and a high concentration of a stressor compound was added to the BLM. The sucrose in the BLM 120 increased the density of the media so that it settled below the TLM110. There is no physical barrier or other separation between the TLM and BLM—only density stratification distinguished the two volumes of media. In the upper region, ports 102 and 104 bring in fresh TLM and remove waste, respectively, at an equal rate. (Pumps 140 and 144 are connected to ports 102, 104, respectively, by sterile tubing.) The BLM 120 was undisturbed by the flow and remained static. Samples were taken from the MICALE chemostat via valves 130, 132, 134 and 136 positioned evenly down the vertical length of the apparatus. These valves divided the media into four sections, each with a distinct microenvironment created by the layering of the TLM and BLM: the permissive layer (PL) 112 in which media is constantly flowing and makes up the majority of TLM 110, the interface (IL) 114, which sits at the now-blurred boundary between the two media types, and the stress layer (SL) 122 below which is the base (BL) 124, which forms the majority of the BLM. The PL 112 represents the area with constant inflow of fresh media and removal of waste and is permissive for wild type growth.

### Media and Stressors

Referring still to FIG. 1, the chemostat involves two layers of different media, separated by density. The top layer media (TLM) 110 was formed of LB broth diluted to 50% to reduce nutrient availability. The bottom layer media (BLM) 120 contained fully concentrated LB broth with 100 g/L of sucrose added so that its density caused it to settle below the TLM which contained no sucrose. To the bottom layer, a high concentration of a stressful compound was added. The appropriate concentration of stressor was determined by growing the ancestor strain on a 96-well microplate in increasing concentrations of the stressor for 24-48 hours, depending on the compound. The determined amount of stressor added to the chemostat was above the observed minimum inhibitory concentration (MIC) of the ancestor. This ensured that any observed growth in the chemostat was due to adaptation rather than a preestablished tolerance of the ancestor strain. In total, three compounds were tested in the BLM during separate runs at concentrations determined to be above the ancestor's tolerance: 100 g/L of sodium chloride (NaCl), 300 mM/L magnesium chloride (MgCl₂), and 120 μg/mL ciprofloxacin (2,000×MIC).

### Multi-Layer Chemostat Set Up and Inoculation

Assembly and use of the MICALE chemostat 100 was performed in a laminar flow hood with a UV light source to sterilize the air and surfaces. In addition to UV-sterilization, all components were wiped down with ethanol prior to use. To assemble the chemostat 100, a round cylinder of thick plastic, e.g., acrylic, polycarbonate, polyvinylchloride, or similar material, was sealed to a base 106 and openings formed in the vertical sidewall for attachment of valves 130, 132, 134 and 136, which are evenly spaced from top to bottom. This assemblage cannot be autoclaved, so it was instead rinsed with Milli-Q water, ethanol, and UV sterilized thoroughly between runs. The top opening was covered with a membrane filter 108 to allow sterile airflow into the chemostat. The valves down the length of the chemostat provide access to different levels within the chemostat. The upper two of these valves (130, 132) could be used as ports for connection to autoclaved tubing ( 1/16^{th} inches inner diameter) through which peristaltic pumps 140, 144 introduced fresh media and removed waste, respectively. Alternatively, as illustrated, separate valved ports 102 and 104, may be used for connection to pumps 140, 144. The media-in tubing connected to port 102 passed through a Sterivex™ filter 118 before being introduced into the chemostat interior. The tubing that removed waste connected from the chemostat to a waste collection bottle 146 outside of the sterile hood. The flow rate of media was tracked by measuring the amount of waste produced over time. To fill the chemostat with media, a peristaltic pump was connected to the lowest valve 136. About 250 mL of TLM was pumped in from the bottom, followed by about 950 mL of BLM so that the two media types remained stacked as the liquid filled the chemostat. The pumps would then be turned on to begin the flow of media in the uppermost layer. The IN-pump 140 drew new media from an autoclaved bottle of 50% diluted LB media. A 0.2-micron filter connected to the lid of the media bottle to allow sterile air to flow in. When the media ran low, freshly autoclaved media (LB) was poured into the stock bottle 142 after the hood was UV sterilized. A 0.2-micron filter on the lid of the stock media bottle allowed sterile airflow. Once the chemostat set up was complete and sterilized with UV, it was shielded from light by covering the assembly with foil, Mylar® film, or other opaque sheet material to prevent future UV damage to cells in the chemostat. E. coli was inoculated to the top of the apparatus using a syringe with 1 mL of the fresh ancestor culture. The time of inoculation was designated as t=0. For illustrative purposes, bacteria are depicted in the figure as wild type 150, in permissive layer 112), adapted strain 152 (in interface layer 114) and adapted strain 154 (in base layer 124).

Sampling from MICALE

During the first 48 hours of a run, samples were taken at intervals of one to three hours during the daytime to track cell growth up to steady state. Afterwards, samples were taken at minimum once every two days. Due to slower growth in the antibiotic run, the sampling frequency was reduced to only twice per day for the first couple days, followed by sampling once a day at most. Sampling was performed by connecting a small peristaltic pump to slowly remove 2 mL of media from each valve to avoid disturbing the density stratification. The small pump was flushed with ethanol after each sample and kept in the sterile hood. At least once a week, an additional 0.5 mL of media was collected and archived as a frozen glycerol stock.

With each sample, the OD₆₀₀, salinity or density, and pH was measured. For OD₆₀₀, 0.2 mL of sample was pipetted in triplicate onto a 96-well plate along with sterile TLM and BLM as controls for a single-time measurement of turbidity. During the high salinity run, the salinity was measured with a refractometer instead of density. These measurements were taken only with the PL and IL samples because the salinity of the lower layers was out of range for the refractometer. For the chaotropic and antibiotic stress runs, density was calculated by measuring 0.1 mL of sample in triplicate. The pH was measured only during the antibiotic run, using a pH probe.

The flow rate was calculated by removing and measuring the amount of media waste in the collection bottle and dividing by the amount of time that had passed since the previous media collection. Time points when the flow was intentionally stopped (to replace a filter, for example) were excluded from the flow rate calculation to prevent the added time from underestimating the flow rate. When the filter clogged and the flow stopped on its own, the media waste was measured, and an estimated flow rate was calculated based on the previous collection. After calculating the estimated flow rate, the rest of the time was recorded as having a flow rate of zero mL per hour. For example, the flow had stopped sometime in the last 24 hours since the last waste collection. The last flow rate calculation was 30 mL/hr and there was now 10 mL of waste in the collection bottle. Since the flow rate could be no faster than the previous 30 mL/hr, the flow was estimated to have continued for at least 3 hours before completely stopping. Thus, the recorded flow rates were 30 mL/hr for three hours and 0 mL/hr for the remaining 21 hours.

Applications, usage and evaluation of the inventive MICALE chemostat may be further understood upon consideration of the following examples, which should not be construed as limiting the scope of the invention in any way.

### Example 1: Bacterial Strain and Culture Conditions

The ancestor culture used in each experiment was from a frozen glycerol stock of Escherichia coli K-12 substrain MG1655 (NCBI: txid511145). Before each experiment, a sample of the frozen ancestor culture was spread onto agar plates with Luria Broth (LB) medium (Lennox) and incubated at 37° C. A single colony was transferred from the plate into 10 mL of liquid LB medium and incubated at 37° C. overnight, or until turbid. 1 mL of this liquid culture was inoculated into the top layer of the MICALE bioreactor, marking the start of the run.

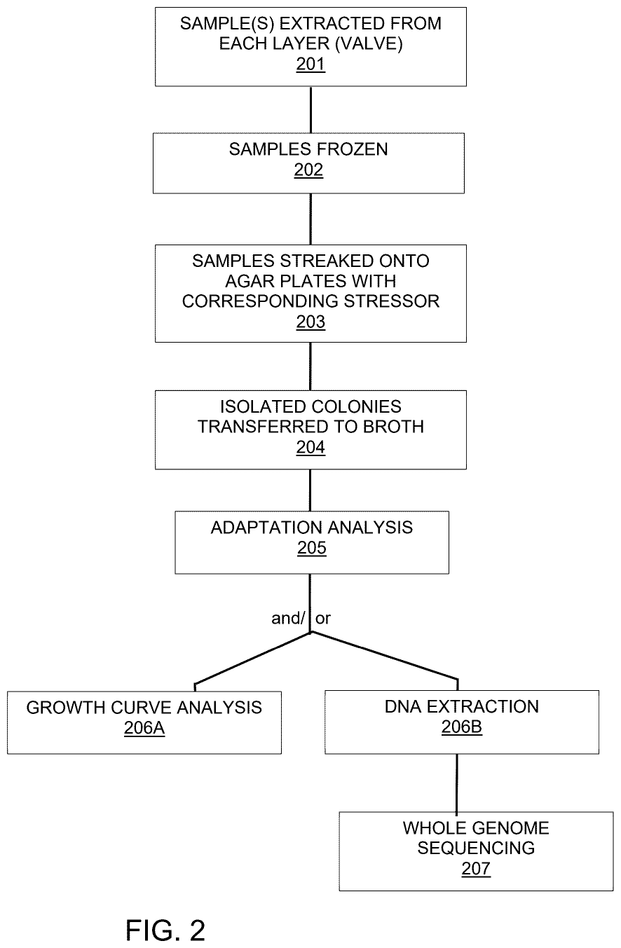

Samples obtained from MICALE runs were cultured before further analysis, an overall schema of which is described in FIG. 2. During runs, samples drawn directly from MICALE (step 201) were frozen at −80° C. with a 1:1 ratio of glycerol (step 202). Frozen samples chosen for analysis were streaked onto LB/agar plates (step 203) and a colony was transferred into 5-10 mL of LB broth medium (in agar plates) to grow a dense culture (step 204). The culture media would often have some level of stressor based on the conditions within the MICALE gradient, such as the type and amount of stressor, and which layer the sample came from. When transferring colonies to broth, the concentration of stressor in the broth matched that in the agar media. Culturing in media with the stressor helped maintain the evolutionary pressure to prevent evolved cells from back-mutating. Samples from the lower, high-stress region were cultured in media that contained a moderate amount of stressor that was low enough to allow growth but high enough to prevent back-mutation. Since the optimal concentration of stressor to achieve growth but prevent back-mutation was not known, samples were grown at multiple concentrations. High salinity stress samples were cultured in media with 0, 40, and 60 ppt NaCl. Chaotropic stress samples were grown in 0, 200, and 250 mM MgCl₂. The antibiotic stress samples were grown in 0, 0.6, and 15 μg/mL ciprofloxacin. Samples were also cultured in blank LB media to observe the colony morphologies of the full bacterial community present in the sample rather than only those selected by one media type.

From the isolated broth cultures in various media types, samples were selected for growth in the adaptation analysis (step 205). The cultures in which media type were chosen for the adaptation analysis based on the culture's ability to produce a turbid culture, what region of MICALE the sample came from, and curiosity. For example, the SL and BL samples used in the adaptation analysis were from cultures of the respective samples in media with the highest level of stressor possible, since the SL and BL samples come from the high-stress BLM region. The PL and IL samples chosen for analysis were generally from the cultures with blank LB media. Sometimes, the PL and IL samples cultured in media with a stressor were used in the analysis to see if adaptation to the stressor could occur in the permissive media. For samples with interesting colony morphologies, multiple (two or more) colonies of different morphology may be used in the analysis to understand how the colony phenotype may correspond with stress tolerance. Adaptation analysis may involve parallel or alternative operations: growth curve analysis (step 206A) and/or DNA extraction (step 206B) and whole genome sequencing (step 207).

### Example 2: Revealing Stress-Tolerant Phenotypes with the Adaptation Analysis

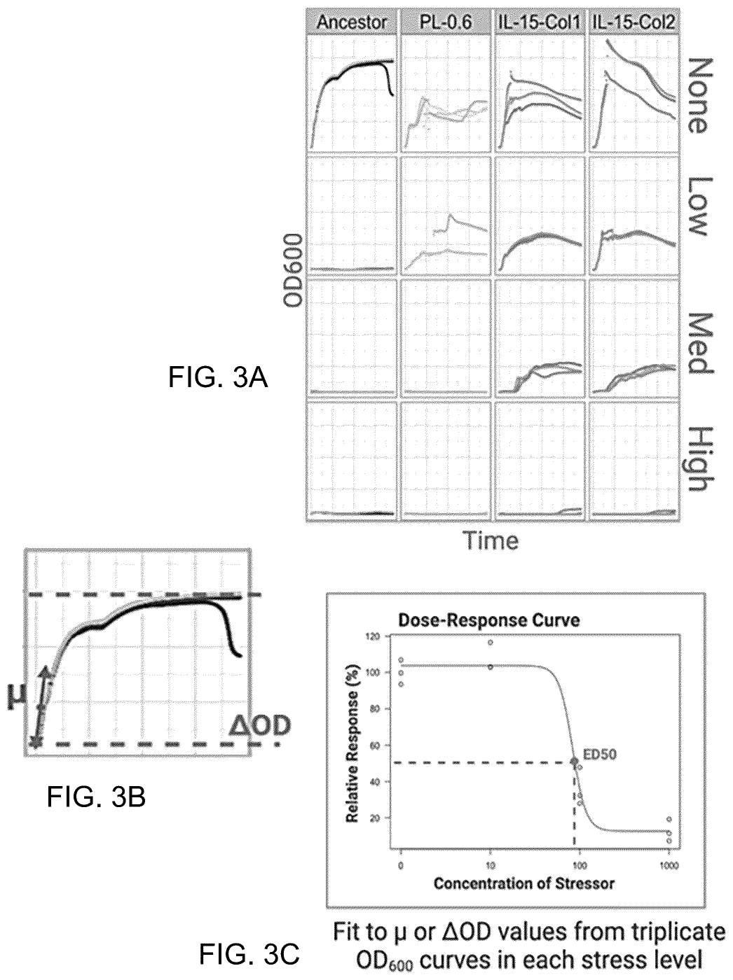

The adaptation analysis (step 205) is a qualitative test to compare the ability to grow in the presence of the stressor between the ancestor and MICALE-grown samples. Turbid broth cultures from isolated colonies were diluted 1:100 in sterile broth with increasing amounts of stressor. These dilutions were vortexed before pipetting 0.2 mL in triplicate onto a 96-well plate. The microplate reader then measured OD₆₀₀ every 15 minutes over the span of four days while incubating the plate at 37° C. with the plate lid on. The measurements resulted in triplicate OD₆₀₀ curves that represent the growth of each sample well on the 96-well plate, shown in FIG. 3A. Each facet of the graph shows the three curves obtained from the triplicate wells of a sample and media treatment. Referring to FIG. 3B, for each OD₆₀₀ curve, the maximum rate of growth over a span of 75 minutes is designated the exponential growth rate, u, and the subtracted difference between the maximum and minimum OD₆₀₀ measurements, AOD, is designated the cell proliferation. The high salinity and chaotropic stress adaptation analyses were completed once whereas the antibiotic analysis was repeated for two total trials. Growth metrics were extrapolated from the growth curves (described below) for numerical comparisons between samples and to fit a logistic dose-response curve of each sample's response in a stressor. For each sample, a logistic curve was fit to the triplicate relative exponential growth rate or relative cell proliferation values across all media types (FIG. 3C). This curve represents a sample's relative response to increasing doses of a stressor, known as a dose-response curve. The value for ED50 is the effective dose of the stressor at which a 50% decrease in the maximal response (100%) is observed.

The samples chosen from the high salinity run were frozen stocks of the PL, SL, and BL samples that had been taken at the end of the run (two weeks) and one SL sample from the first 25 hours (Table 2, below). The samples used in the adaptation analysis had been in media with a NaCl concentration of 60 ppt, and the ancestor was cultured on blank LB media. The samples were diluted into LB media with 5, 45, 60, 75, and 90 ppt NaCl then plated on the 96-well plate. Normal LB media with no additional salt added had an NaCl concentration of 5 ppt.

For the chaotropic stress run, samples from both two weeks and four weeks into the run were selected for analysis (Table 5, below). The samples, including the ancestor, were cultured in media with 0, 200, and 250 mM MgCl₂. The IL samples were not plated on 200 mM MgCl₂ plates due to limited resources. The ancestor was grown on plates with the same MgCl₂ concentration as the samples to see if the culturing process was causing genetic change significant enough to affect the growth phenotypes observed during the adaptation analysis. Cultures were diluted into media with 0, 200, 250 and 300 mM MgCl₂ then pipetted onto the 96-well-plate to start the four-day analysis.

The samples chosen for the antibiotic-stress adaptation analysis were from the end of the antibiotic stress MICALE run, sampled on the 14th day (Table 7, below). Samples were cultured grown in media with no antibiotic, 0.6 μg/mL ciprofloxacin (10×MIC,) and 15 μg/mL ciprofloxacin (250×MIC) were transferred into 5 mL of LB broth with the same amount of antibiotic. The only exception is the BL sample was grown in 0.6 μg/mL ciprofloxacin then transferred to LB broth with 15 μg/mL ciprofloxacin. Once turbid, the broth cultures were diluted into media with no antibiotic, 0.6 μg/mL, 6 μg/mL, and 60 μg/mL ciprofloxacin then pipetted onto a 96-well plate and placed in the microplate reader to start the analysis. A portion of the broth cultures used for the analysis were frozen in glycerol. For a repeat trial of the adaptation analysis, these frozen stocks were revived in broth media matching the level of antibiotic in which they had originally been cultured.

### Example 3: Exponential Growth Rate and Relative Cell Proliferation

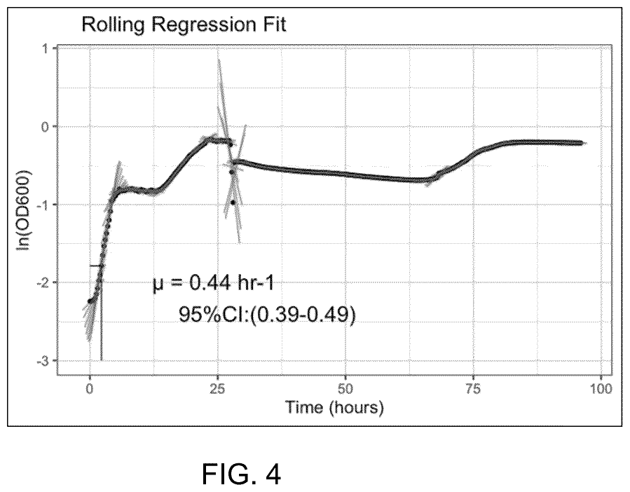

To statistically compare growth between samples, two parameters were extracted from the OD₆₀₀ curves resulting from the adaptation analysis: relative exponential growth rate and relative cell proliferation (FIG. 3B). To find the exponential growth rate, μ, growth curves are normally fit to a standard sigmoidal function, such as the Gompertz model. However, these OD₆₀₀ curves were too irregular to be well fit with a standard function. Instead, a rolling regression was fitted onto natural log transformed OD₆₀₀ curves to produce linear regression lines between six measurements at a time (75 total minutes), shifting by 1 measurement, over the curve. The exponential growth rate from the maximum slope obtained by the rolling regression is 0.44 hr⁻¹. The darker lines on the upper right section of the curve represent where the maximum slope occurs. (FIG. 4).

The exponential growth rate was determined to be the maximum slope of any of the regression lines, denoted by μₛₜᵣₑₛₛ for growth curves in media with stressor and μ_{blank} for that in blank LB media [Equation 1]. Any slope maxima that appeared to be due from anomalies in the data, such as a quick jump in OD₆₀₀ after biofilm formation, were filtered out. This ensured that the maximum slope occurred during the time that visually represented exponential growth (near the start of the curve). The triplicate μ_{blank} calculations were averaged to obtain the values representing that sample's growth in media without stress, μ̅_{control} [Equation 2]. The percent relative exponential growth rate was obtained by dividing each μ by the μ̅_{control}, then multiplying by 100 [Equation 3]. Finally, the three relative exponential growth rate values for each sample and media type were averaged [Equation 4].

$${μ}_{stress or blank} = max slope of f ( time , ln ( {OD}_{600} ) ) \qquad [ 1 ]$$

$${\overline{μ}}_{c o n t r o 1} = \frac{{μ}_{b l a n k 1} + {μ}_{b l a n k 2} + {μ}_{b l a n k 3}}{3} \qquad [ 2 ]$$

$$\% Relative Exponential Growth Rate = 100 \times \frac{{μ}_{s tress or blank}}{{\overline{μ}}_{control}} \qquad [ 3 ]$$

$$Average \% Relative Exponential Growth Rate = 100 \times [ ( \frac{{μ}_{s t r e s s 1} + {μ}_{s t r e s s 2} + {μ}_{s t r e s s 3}}{{\overline{μ}}_{c o n trol}} ) ] \qquad [ 4 ]$$

Relative cell proliferation was calculated for each OD₆₀₀ curve (three curves per media type per sample) first by finding ΔODₛₜᵣₑₛₛ, the subtracted difference for each curve grown in media with the stressor and that grown in blank media, ΔOD_{blank} [Equation 5]. Any OD₆₀₀ curves that showed an abnormal jump and fall in optical density, caused by variables such as biofilm formation, were filtered out so that ΔOD was calculated using OD₆₀₀ values that more accurately represented the actual cell count. Each sample's three ΔOD_{blank} calculations were averaged to obtain the Δ̅O̅D̅_{control} [Equation 6]. The percent relative cell proliferation was calculated by dividing the ΔOD by Δ̅O̅D̅_{control} and multiplying by 100 [Equation 7]. Lastly, the three relative cell proliferation values for each sample and media type were averaged [Equation 8].

$$Δ {OD}_{s tress or blank} = {OD}_{max} - {OD}_{min} \qquad [ 5 ]$$

$${\overline{Δ OD}}_{c o n trol} = \frac{Δ O {D}_{b l a n k 1} + Δ O {D}_{blank 2} + Δ O {D}_{b l a n k 3}}{3} \qquad [ 6 ]$$

$$\% Relative Cell Proliferation = 100 \times \frac{Δ O {D}_{s tress or blank}}{{\overline{Δ OD}}_{c o n trol}} \qquad [ 7 ]$$

$$Average \% Relative Cell Proliferation = 100 \times [ ( \frac{Δ {OD}_{s t r e s s 1} + Δ {OD}_{s t r e s s 2} + Δ {OD}_{s t r e s s 3}}{{\overline{Δ OD}}_{c o n trol}} ) ] \qquad [ 8 ]$$

### Example 4: Dose-Response Curves and Effective Dosage (ED) Calculations

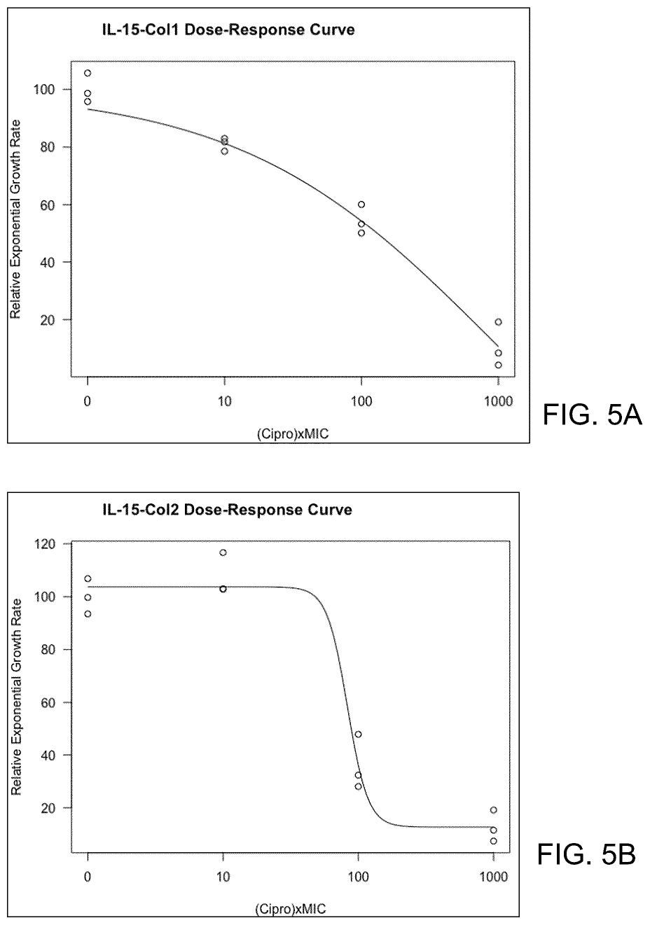

Using the exponential growth rate and relative cell proliferation values derived from the OD₆₀₀ growth curves, dose-response curves were fit to understand how sensitive each sample strain was to an increase in stressor concentration (FIG. 3C and FIGS. 5A-5B). The dose-response curves were generated using the drc R package, modeled with the four-parameter logistic function given by:

$$f ( x , ( b , c , d , e ) ) = c + \frac{d - c}{1 + exp \{ b ( log ( x ) - log ( e ) ) \}} \qquad [ 9 ]$$

The parameters d and c represent the upper limit and lower limit of the logistic curve, respectively. The parameter e, also referred to as ED50, is the dosage that occurs half-way between d and c and can be calculated using Equations [10] and [11]. The logistic function is symmetrical around e. The fourth parameter, b, is the relative slope around e, also known as the Hill coefficient.

EDy is the effective dosage quantity at which the maximal response, d, is decreased by y %, yielding a response that is (100-y %) of d [Equation 10]. Since the model is symmetrical around e, the amount that dis decreased by, y percent, is a proportion of the total distance between d and c. Note that the distance between d and c is not the distance between d and 0.

$$EDy = {e ( \frac{y}{100 - y} )}^{\frac{1}{b}} \qquad [ 10 ]$$

$$ED 50 = {e ( 1 )}^{\frac{1}{b}} \qquad [ 11 ]$$

The ED50 dosage for each sample serves as a metric for sensitivity to the stressor that can be compared between samples. However, when this model was applied to the data, the logistic fit sometimes resulted in a negative lower limit, c (FIG. 5B). This produced an over-estimated value for ED50 since the 50% decrease was measured by the distance between d and c and not between the observed maximum and minimum response values of the data.

To evaluate the effect of increasing antibiotic (ciprofloxacin) concentration, dose-response curves were fit to the relative exponential growth rate in units of minimum inhibitory concentration (MIC) for IL-15-Col1 sample (FIG. 5A) and IL-15-Col2 sample (FIG. 5B) grown during trial 1 of the antibiotic adaptation analysis. The lower limit of the logarithmic fit, c, for FIG. 5A is −75.5, producing the convex-curve shape instead of the expected biological growth curve seen in FIG. 5B in which the lower limit is 12.7. For FIG. 5A, the unadjusted ED50 value shows a ciprofloxacin concentration of 926.79×MIC, which seemed quite high and did not correlate to what was observed in the data. The ED50_{adjusted} value is 130.13×MIC, which more closely aligns with a 50% decrease from the maximum growth rate. For FIG. 5B, the unadjusted ED50 value is 103.73×MIC and the ED50_{adjusted} is 87.29×MIC.

To account for the over-estimated ED50 values, new ED50_{adjusted} values were calculated using the y percentage that represented a decrease of d that is half the value of d [Equations 12, 13]. The resulting EDy was plugged into the drc package that calculated the concentration of media at which the response was decreased by d/2. This adjustment was made across all dose-response models so that all reported ED50 values represent a 50% decrease in the maximum response.

$$y = 100 \times \frac{\frac{d}{2}}{( d - c )} \qquad [ 13 ]$$

$$ED {50}_{adjusted} = EDy = {e ( \frac{100 \times \frac{\frac{d}{c}}{( d - c )}}{100 - ( 100 \times \frac{\frac{d}{2}}{( d - c )} )} )}^{1 / b}$$

The ED50 was calculated for both the relative exponential growth rate and cell proliferation dose-response curves for each sample. Some samples could not be fit with a dose-response curve because they grew poorly or not at all in the stressor, thereby providing insufficient depth of data points for the logistic function to be fit. This problem occurred especially for the antibiotic samples that could not grow in ciprofloxacin, such as the ancestor. Samples that could not be fit to a model were excluded from ED50 analysis.

To obtain ED50 values for the ancestor strain in ciprofloxacin, the dose-response curves were fit to the relative exponential growth rate and cell proliferation values obtained from the MIC growth test in which the ancestor was grown in smaller increments of ciprofloxacin thereby providing enough resolution to fit a logistic function. It is recognized that the MICALE samples were grown on a much larger, low-resolution scale of ciprofloxacin, resulting in ED50 values that are less precise. However, the samples clearly grew in the presence of ciprofloxacin, unlike the ancestor. Thus, for testing purposes, the ED50 values were seen as an accurate representation of the observed difference in antibiotic resistance between MICALE samples and the ancestor.

### Example 5: Statistical Analysis

Differences between the density of the sample layers and control media types were assessed using a pairwise Welch two-sample t-test in RStudio with unpaired data, unequal variance, non-pooled standard deviations, and no p-value adjustment options. Statistical analyses were not performed with any ED50 values because the ED50 sample size was too small to allow for a calculation of significance. Future MICALE runs with more replicates should perform a t-test to calculate the significance of the ED50 values.

### Example 6: DNA Extraction and Sequencing

Two rounds of DNA extraction were performed with different protocols. The first round extracted DNA from the high salinity and chaotropic stress experiments and the second round extracted from the antibiotic experimental samples. For the high salinity and chaotropic stress experiments, frozen samples and ancestors of interest were spread onto LB/agar plates with a concentration of stressor matching their origin: blank LB/agar, 60 ppt NaCl LB/agar, or 200 mM MgCl₂. The frozen high salinity samples had come directly from MICALE. The frozen chaotropic stress samples were from broth cultures of the isolated colonies that were grown up for use in the adaptation analysis. Colonies from the agar plate cultures were inoculated into broth with the same concentration of stressor. Once turbid, 2 mL of broth culture was centrifuged, and the resulting pellet transferred into a new tube with sterile Milli-Q water. DNA extraction was performed using the Zymo Quick-DNA Fungal/Bacterial Miniprep Kit. Afterwards, the DNA concentration was measured with a Qubit. Samples with DNA concentrations less than 2 μg/uL were extracted again.

For the antibiotic experiment, frozen samples and ancestors of interest were transferred directly into sterile broth with a concentration of ciprofloxacin of 0, 0.6, or 15 μg/mL. It was not necessary to isolate a colony prior to the broth culture because these frozen stocks had come from the isolated cultures grown up for the adaptation analysis, rather than samples straight from MICALE. After incubating for two days at 37° C., 1.5 mL of the culture was transferred into a 2 mL Eppendorf tube and centrifuged. The resulting pellet was extracted into an effluent sample using the MagMAX Microbiome Ultra Nucleic Acid Isolation Kit, followed by DNA extraction with the KingFisher Flex system, and DNA concentration readings with a Qubit. Library preparation from the extractions used standard protocols and primers, and an Illumina NovaSeq6000 was used for whole genome sequencing Referring briefly back to FIG. 2, these operations correspond to steps 206B and 207, respectively.

### Example 7: Identification of Genetically Evolved Strains

Whole-genome resequencing analysis was performed using the breseq computational pipeline (Barrick Lab, University of Texas, Austin, TX) to identify mutations present in MICALE-grown samples that were distinct from the ancestor. ILLUMINA® forward and reverse reads were quality filtered to remove reads with a quality score less than 30 using the sequence preprocessor fastp and checked the results with FastQC. The filtered reads were mapped in breseq to the E. coli K-12 MG1655 reference genome accessed from the National Center for Biotechnology Information (Accession no. GCA_000005845.2). Out of the 24 samples sequenced from the high salinity and chaotropic stress runs, six samples had a mapped percentage less than 98%. All antibiotic samples had mapped percentages of less than 5%. Low mapped percentages indicated the sample identity was not E. coli MG1655. To identify the samples, the first and last ten lines of the fastq files were manually changed to fasta format then inputted the fasta sequences into a GenBank BLAST search. Each search match was a bacterial genus different from E. coli, indicating that these samples were contaminated. For the non-contaminated MICALE-derived sequences, mutations identified by breseq were compared to the mutation results for the ancestor strain used to inoculate each run. Mutations overlapping with the ancestor were removed to reveal only mutations distinct from the ancestor.

### Example 8: Spatial Gradient of Stress in MICALE

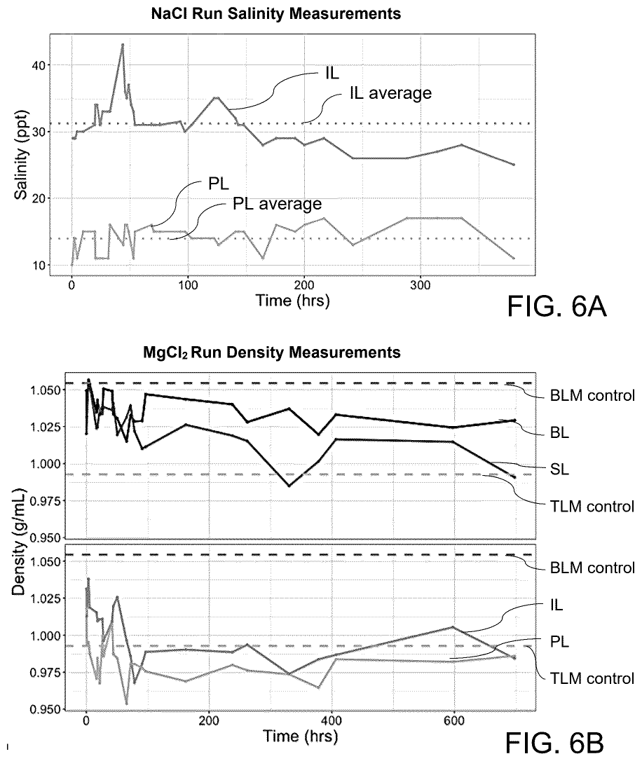

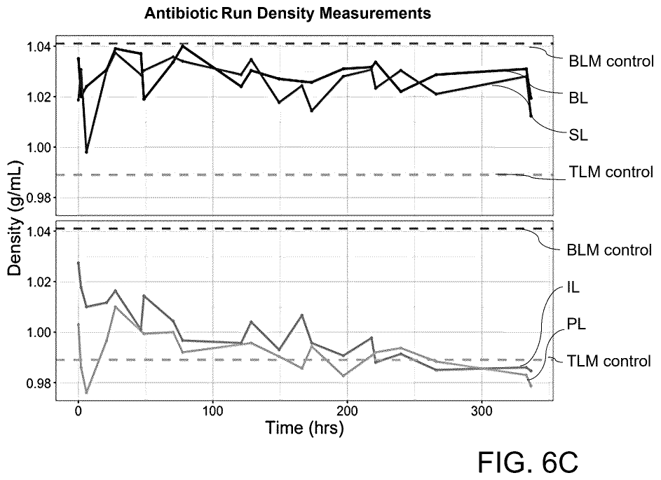

Density or salinity measurements during each MICALE run indicated that a difference in density between the upper and lower regions was maintained throughout the run. In the high salinity run, the measured salinity of samples from the PL (permissive layer) and IL (interface layer) valves remained constant and separated (FIG. 6A). The refractometer salinity of samples from the IL layer, where top-layer media (TLM) meets bottom-layer media (BLM), stayed above 29 ppt NaCl for the run duration with an average salinity of 31 ppt NaCl. The PL samples, where media is majority TLM, stayed below 16 ppt NaCl with an average salinity of 14 ppt NaCl. A two-sample t-test compared the salinity of the two layers returns a p-value of 2.2e-16, confirming that the salinity of the two sampling valves is significantly different, despite the PL and IL valves being relatively close to each other. Results are shown in the upper section of Table 2.

For the chaotropic stress and antibiotic runs, the density of each layer fluctuated but overall remained separated and distinct (FIG. 6B). During the chaotropic stress run, the density of the IL, SL, and BL decreased slightly over time while the PL density stayed relatively constant. There were noted fluctuations in the density measurements in the first 100 hours that appear to be somewhat similar across the four layers. Despite these fluctuations in density, the PL and SL sample measurements from the chaotropic stress run never crossed; the SL was always denser than the PL for the entire month-long run.

For the chaotropic stress run, the average density of the PL, IL, SL, and BL layers was 0.9821, 1.0006, 1.0235, 1.0363 g/mL, respectively. The measured density of sterile and separated TLM and BLM controls was 0.9923 and 1.0551 g/mL, respectively. We performed a pairwise independent t-test comparison of all the densities, the full results of which can be viewed in the center rows of Table 2. Our t-test showed the PL and IL density measurements were statistically different from the BLM control, the SL layer, and BL layer measurements with p-values less than 0.05. On the other hand, the PL and IL measurements were not distinct from the TLM control measurements with p-values greater than 0.05. Similarly, the BL densities were statistically indistinct from the BLM control but different from the TLM control. This was expected since media from the bottom of MICALE should be the densest and most static. The SL sample densities, on the other hand, are statistically different from both the TLM and BLM controls, as well as different from the other sampling layers. Despite there being no physical barrier between the TLM and BLM, the PL and IL sampling layers remained indistinct from the TLM and remained statistically different from the denser media in the SL, BL, and BLM samples. As in the high salinity run, the densities of adjacent sampling valves were statistically different: the PL and IL densities differ with a p-value of 5.4e-08 and IL and SL densities with a p-value of 2.7e-10.

Similar to the chaotropic stress run, the density measurements from the antibiotic run fluctuated but remained distinct between layers (FIG. 6C and Table 2, lower rows). Sterile TLM and BLM media, before being added to MICALE, had respective densities of 0.9890 and 1.041 g/mL. The average density of the PL, IL, SL, and BL samples was 0.9921, 1.001, 1.025, and 1.029 g/mL, respectively. When compared for statistical difference, the PL densities are similar to the TLM control, and the SL and BL densities are similar to the BLM control and to each other. The IL sample densities are statistically different from both the TLM and BLM controls and each of the three other samples, like the SL densities from the chaotropic stress run.

TABLE 2
NaCl Salinity T tes

| | PL |
|---|---|
| IL | 2.2e−16 |

MgCl₂ Density t test

| | TLM | BLM | PL | IL | SL |
|---|---|---|---|---|---|
| BLM | 0.00045 | — | — | — | — |
| PL | 0.28787 | 0.00030 | — | — | — |
| IL | 0.37448 | 0.00121 | 5.4e−08 | — | — |
| SL | 0.01203 | 0.01531 | <2e−16 | 2.7e−10 | — |
| BL | 0.00269 | 0.08984 | <2e−16 | <2e−16 | 5.7e−05 |

Antibiotic Density t test

| | TLM | BLM | PL | IL | SL |
|---|---|---|---|---|---|
| BLM | 0.02250 | — | — | — | — |
| PL | 0.49279 | 0.03213 | — | — | — |
| IL | 0.04237 | 0.04749 | 0.00276 | — | — |
| SL | 0.00069 | 0.22868 | <2e−16 | 6.2e−11 | — |
| BL | 0.00071 | 0.32574 | <2e−16 | 1.1e−14 | 0.24006 |

### Example 9: Cell Growth, OD₆₀₀ Measurements, Flow Rate, and pH

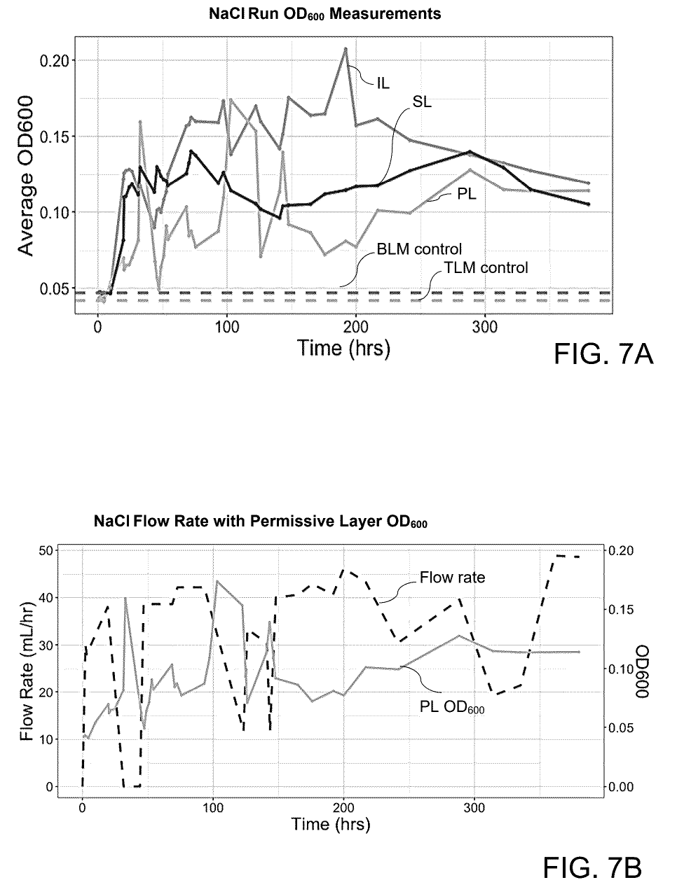

OD₆₀₀, flow rate, and pH measurements with observed cell growth characteristics provided information on the dynamics of E. coli growth in the MICALE apparatus. For the high salinity stress run, the measured OD₆₀₀ of the three sampled layers, the PL, IL, and SL, all increased within the first 25 hours (FIG. 7A). Afterwards, the OD₆₀₀ of the IL and SL samples stayed relatively stable, compared to the PL that spiked and dipped regularly. The media flow during the high salinity run fluctuated regularly, mostly resulting from mechanical pump issues (FIG. 7B). When compared with the measured flow rate, the OD₆₀₀ of the PL spiked at times when the flow rate dropped off and stabilized when the flow rate was constant. The OD₆₀₀ of the other samples did not show a similar trend with flow rate. Lastly, it is notable that cultures of the high salinity stress MICALE samples took a long time to grow. Even when incubated at 37° C., agar plates of the samples took over four days to grow small colonies.

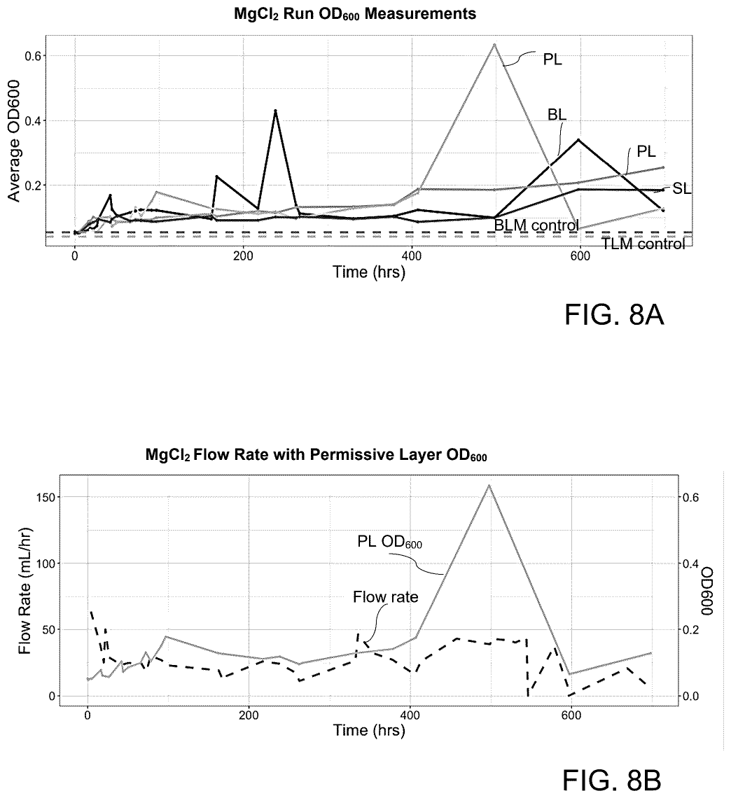

For the chaotropic stress run, the OD₆₀₀ of the PL, IL, SL, and BL layers increased within the first 25 hours then began to stabilize (FIG. 8A). The OD₆₀₀ for the PL and BL sample layers frequently spiked, as seen in the PL OD₆₀₀ at 500 hours, for example. These spikes occurred when goopy aggregates, suspected to be extracellular polysaccharides (EPS) were collected in the sample vials thereby altering the turbidity of the liquid sample and producing a high OD₆₀₀ measurements. The majority of these cell aggregates were seen near the top of MICALE, attached to the walls and collected at the bottom of MICALE. Consequently, the PL and BL OD₆₀₀ measurements were affected the most, causing the spikes seen in FIG. 8B. These spikes in the data make it difficult to draw comparisons between OD₆₀₀ and the flow rate.

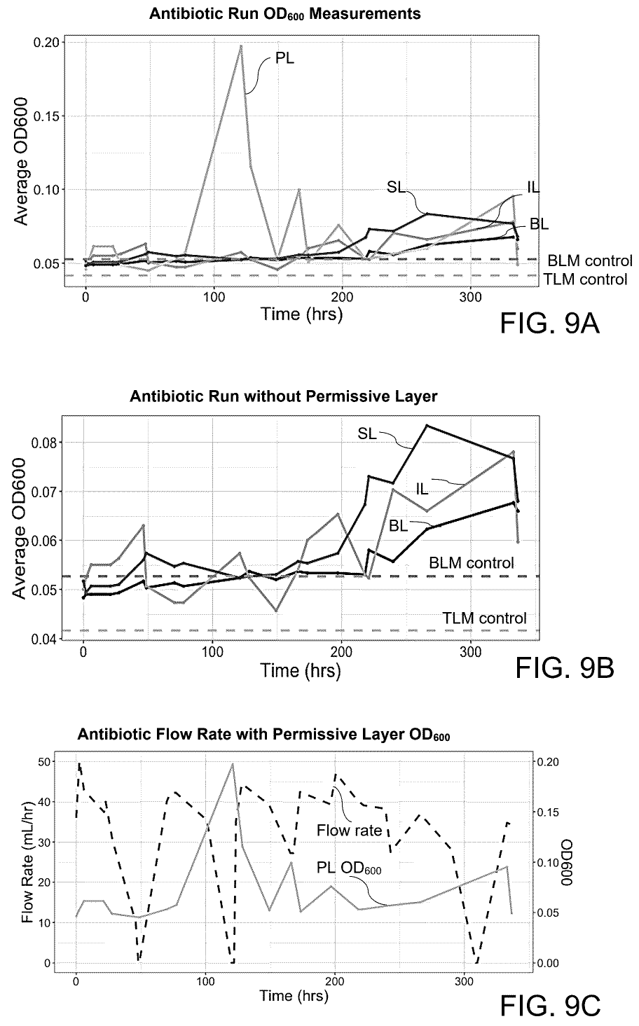

The OD₆₀₀ measurements during the antibiotic run were much lower than the other runs (FIGS. 9A, 9B). The IL, SL, and BL layer OD₆₀₀ rose to just over 0.08 absorbance in the last 200 hours, compared to the high salinity and chaotropic stress runs that reached well over 0.1 absorbance in the first 48 hours. Meanwhile, cells grew in high densities in the in-valve and tubing that fresh media flowed through, clogging the filter; cells were escaping from the ciprofloxacin inside MICALE, following the flow of antibiotic-free media. Like the high salinity run, the antibiotic PL OD₆₀₀ peaked during times when the flow rate dropped, for example at around 125 hours there is a large spike in PL OD₆₀₀ when the flow rate had all but stopped due to mechanical issues (FIG. 9C). OD₆₀₀ measurements for all antibiotic sample layers rose in the last 200 hours then appear to drop by the final sample. This drop is also seen in the final density measurements. It is important to note that the day prior to the final sample, when the OD₆₀₀ measurements appeared to rise, it was observed that the filter had clogged sometime in the previous 48 hours and there was no media flow. The filter was fixed to allow media to flow for another 24 hours before the final sample was taken. The return of the flow rate is presumably what decreased the cell density (OD₆₀₀ measurements) by the time of the final sample.

Noting that waste from the salinity and chaotropic stress runs smelled fermented, we measured pH during the antibiotic run because we suspected that cells may be undergoing anerobic fermentation. The starting pH measurements for the antibiotic stress samples were 6.93, 6.93, 6.90, and 6.95 for the PL, IL, SL, and BL samples. The pH measurements for the lower two valves stayed between 6.7 and 7 at the start of the run. However, beginning at around 125 hours, the pH in the IL started to decrease, followed by the other layers at around 200 hours. The PL layer decreased somewhat but continued to fluctuate between 6.1 and 6.7. The IL, SL, and BL layers, on the other hand, decreased sharply. The IL sample region had the lowest pH measurement of 5.45 at 266 hours. The final pH measurements at the end of the run for the PL, IL, SL, and BL samples were 6.6, 5.74, 5.67, and 6.28, respectively.

### Example 10: High Salinity Adaptation Analysis

During the high salinity adaptation analysis, isolated MICALE samples grew in a higher concentration of NaCl than the ancestor. The samples grown in the adaptation analysis were three samples from the end of the run, PL, SL_{final}, and BL, one sample from the first 25 hours, SL_{early}, and the Ancestor, as shown in Table 3.

TABLE 3

| | | Time of | | |
|---|---|---|---|---|
| Name in | | Sample | NaCl in Culture | |
| Analysis | Sample Origin | (days) | Media (ppt) | Breseg Results |
| Anc | Ancestor | — | 5 | Fully mapped |
| SL_{early} | Stress Layer | 1 | 60 | Partially contaminated: E. coli and |
| | | | | Bacillus |
| PL | Permissive | 14 | 60 | Contaminated: Bacillus |
| | Layer | | | |
| SL_{final} | Stress Layer | 14 | 60 | Contaminated: Bacillus |
| BL | Base | 14 | 60 | Fully mapped: no distinct mutations |
| PL-05 | Permissive | 14 | 5 | Fully mapped: no distinct mutations |
| | Layer | | | |
| IL-05 | Interface | 14 | 5 | Fully mapped: three mutations |
| IL-60 | Interface | 14 | 60 | Contaminated: Bacillus |

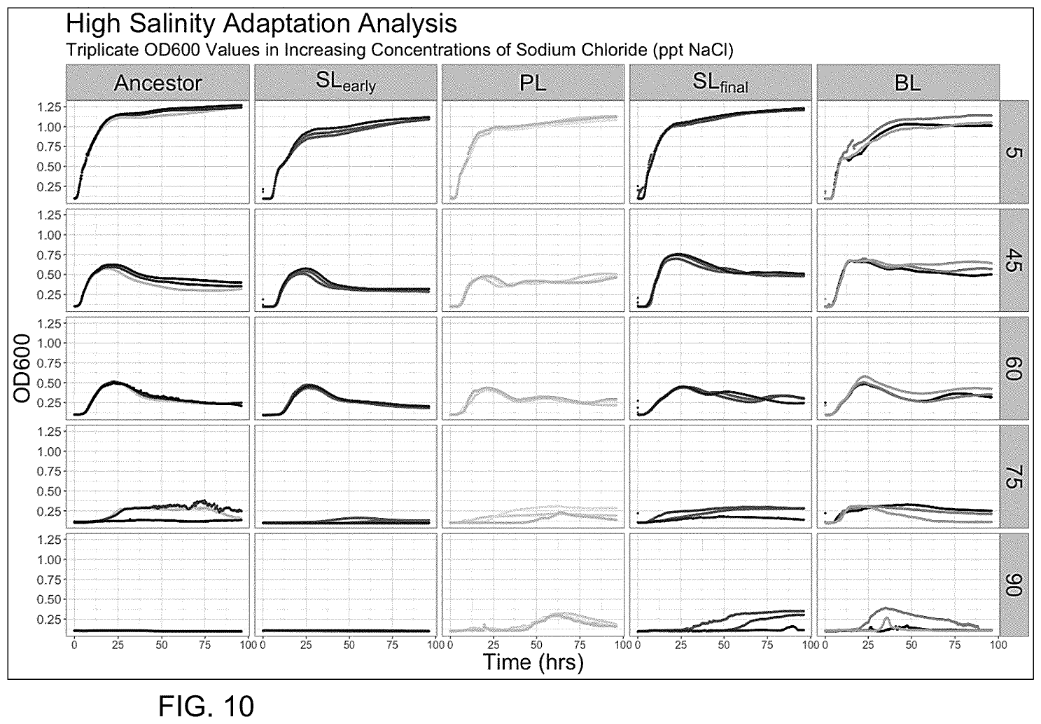

The upper five rows of the table above the dark line identify samples used in the adaptation analysis, shown in FIG. 10. The lower three rows correspond to samples that were only sequenced. Before the adaptation analysis and DNA extraction, colonies were selected from the samples grown on media with different concentrations of NaCl, indicated by the “NaCl in Culture Media (ppt)”. The column for breseq results indicates if the sample was fully mapped to the E. coli reference genome or contaminated. For fully mapped samples, the number of distinct mutations, if at all, is listed.

Over four days these samples were grown in triplicate in media with salinity concentrations of 5, 45, 60, 75, and 90 ppt NaCl. The three samples taken at the end of the MICALE run showed growth in every level of salinity with all average cell proliferation values over 5%. For each sample the cell proliferation, denoting the amount of biomass, decreased with increasing salinity, except for the PL and SL_{final} that increased their average cell proliferation values from 75 to 90 ppt NaCl. The Ancestor and SL_{early} sample reached average cell proliferation values of less than 5% in 90 ppt NaCl and 75 and 90 ppt NaCl, respectively, denoting no growth.

There was remarkable variation in the shape of the OD₆₀₀ curves between samples, especially in higher salinities, as seen in FIG. 10. Between samples, the time at which exponential growth occurred, the rate of exponential growth, and total amount of cellular biomass differed, forming unique growth curve shapes. For all samples, the average exponential growth rate decreased in similar fashion from 5 to 60 ppt NaCl, except for the PL sample that did not decrease in 60 ppt NaCl. Starting at 75 ppt NaCl, however, we observed greater variation in average exponential growth rate as the samples decreased or increased their growth rate different amounts: from 60 to 75 ppt NaCl, the PL, SL_{early}, and Ancestor decreased their exponential growth rate by 35.0, 28.9, and 22.9 percent, respectively, whereas the SL_{final} decreased by only 7.8 percent and the BL increased by 7.7 percent. The resulting relative growth rate for the PL, SL_{early}, Ancestor, SL_{final}, and BL was 18.3, 3.1, 15.5, 19.7, and 40.2 percent. Some samples decreased their exponential growth rate with increasing salinity whereas others maintained or even increased their exponential growth rate.

In 75 ppt NaCl, the SL_{final} and PL produced curves with varying steepness but overall had longer and lower slopes and very similar exponential growth rates. The Ancestor and BL sample, on the other hand, had more defined areas of exponential growth and a leveled-off stationary phase. Starting just before 50 hours, the OD₆₀₀ measurements for one Ancestor measurement in 75 ppt NaCl looked fuzzy, indicating instrumental noise likely from an external factor like evaporation. These measurements were filtered out before statistical analysis. In 90 ppt NaCl, the curves for the PL, SL_{final}, and BL samples were shorter, showing signs of growth much later in time. The SL_{final} and BL samples in 90 ppt showed a lot of variation within the triplicate wells. Abnormal, small spikes in the data, such as that seen at around 30 and 90 hours for the BL and SL_{final}, respectively, could not be ruled out as instrument noise and were included in further analysis.

The BL sample's average exponential growth rate increased from 60 to 75 and to 90 ppt NaCl with relative values of 32.5, 40.2, and 42.8 percent, respectively. On the other hand, the relative cell proliferation of the BL sample decreased as salinity increased; in higher salinities, the BL sample reached higher maximum growth rates but lower overall biomass. The PL and SL_{final} sample reached their lowest maximum growth rate in 75 ppt with percent responses of 18.3%, and 19.7%. In 90 ppt NaCl, both samples increased their exponential growth rates to 25.9% and 27.0%. The ancestor's response was more typical, producing its lowest exponential growth rate and cell proliferation values in the highest concentration of salinity.

The ED50 values from the NaCl adaptation analysis, listed in Table 4, showed nearly an inverse relationship between the ranking of sample's ED50s for exponential growth rate and cell proliferation.

TABLE 4

| | Relative Growth | Relative Cell |
|---|---|---|
| Sample Name | Rate ED50 | Proliferation ED50 |
| Ancestor | 53.5 | 40.3 |
| SL_{early} | 36.4 | 44.2 |
| PL | 50.9 | 36.0 |
| SL_{final} | 48.8 | 48.3 |
| BL | 46.0 | 52.7 |

The Ancestor had the highest ED50 calculated from the exponential growth rate, with a 50% decrease in the max growth rate occurring at a salinity of 53.5 ppt NaCl. On the other hand, the Ancestor had the lowest cell proliferation ED50 value of 40.3 ppt NaCl, excluding the PL sample which had a very low value. In contrast to the Ancestor, the BL sample had the lowest growth rate ED50 value of 46.0 ppt NaCl and highest cell proliferation ED50 of 52.7 ppt NaCl, excluding the SL_{early} and PL anomalies. The SL_{final} sample had similar ED50 concentrations of 48.8 and 48.3 for growth rate and cell proliferation, respectively. The SL_{early} strain had the lowest growth rate ED50, matching the fact that it was the only sample unable to grow in 75 ppt NaCl. The PL sample had a very low cell proliferation growth rate ED50 of 33.0, compared to its growth rate ED50 of 50.9. These ED50 values matched the trends extracted from the PL curves: from 60 to 75 ppt NaCl, the PL sample's average cell proliferation decreased whereas the average exponential growth rate stayed nearly the same.

### Example 11: Chaotropic Stress Adaptation Analysis

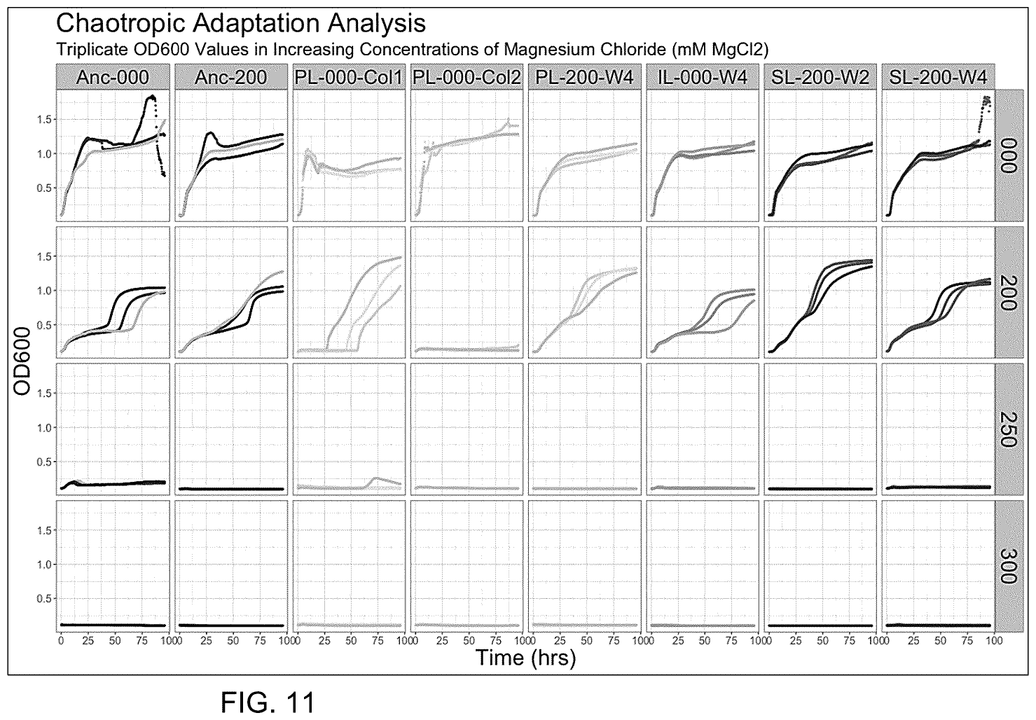

FIG. 11 shows the results of the chaotropic stress adaptation analysis in which the samples were grown for four days in 0, 200, 250, and 300 mM MgCl₂. The samples selected for the MgCl₂ adaptation analysis can be found in Table 5.

TABLE 5

| | | Time of | MgCl₂ in | |
|---|---|---|---|---|
| Name in | | Sample | Culture Media | |
| Analysis | Sample Origin | (weeks) | (mM) | Breseg results |
| Anc-000 | Ancestor | — | 0 | Fully mapped |
| Anc-200 | Ancestor | — | 200 | Fully mapped |
| PL-000-Col1 | Permissive | 4 | 0 | Contaminated: Pseudomonas |
| | Layer, colony 1 | | | |
| PL-000-Col2 | Permissive | 4 | 0 | Contaminated: Pseudomonas |
| | Layer, colony 2 | | | |
| PL-200-W4 | Permissive Layer | 4 | 200 | Fully mapped: no distinct mutations |
| IL-000-W4 | Interface | 4 | 0 | Fully mapped: no distinct mutations |
| SL-200-W2 | Stress Layer | 2 | 200 | Fully mapped: one mutation, missing 2 |
| | | | | mutations |
| SL-200-W4 | Stress Layer | 4 | 200 | Fully Mapped: one mutation |
| PL-000-W2 | Permissive Layer | 2 | 0 | Fully mapped: no distinct mutations |
| PL-200-W2 | Permissive Layer | 2 | 200 | Fully mapped: no distinct mutations |
| IL-000-W2 | Interface | 2 | 0 | Fully mapped: no distinct mutations, |
| | | | | missing 1 mutation |
| BL-000-W2 | Base | 2 | 0 | Fully mapped: no distinct mutations |
| BL-200-W4 | Base | 4 | 200 | Fully mapped: three mutations |

The upper eight rows of Table 5 (above the dark line) correspond to the samples used in the adaptation analysis shown in FIG. 11. The samples listed in the lower five rows were sequenced only. The name of the sample indicates where the sample came from (Ancestor, PL, IL, SL, or BL,) the concentration of MgCl₂ in which the sample was cultured before analysis (000 or 200 mM,) and the time that the sample was taken (two weeks or four weeks.) Samples PL-000-Col1 and PL-000-Col2 indicate separate colonies with different morphologies from the same culture plate. The column for breseq results describes if the sample was fully mapped to the E. coli reference genome or contaminated. For fully mapped samples, the number of distinct mutations, if at all, is listed.

No samples showed signs of growth in 300 mM MgCl₂, the starting concentration of magnesium chloride in the BLM of MICALE. Most of the samples did not grow in 250 mM MgCl₂ either. Only the Anc-000 and PL-000-Col1 showed signs of growth in 250 mM MgCl₂; The PL-000-Col1 had one well that showed a rise then fall of OD₆₀₀ in the last 60 hours of the run. The Anc-000 triplicate wells in 250 mM MgCl₂ had an early bump and fall in OD₆₀₀ in the first 24 hours then slowly rose over time (FIG. 11, third row). While the bump may be due to growth, the slow rise following the bump could be the result of evaporation. Anc-000 was plated on the edge of the 96-well plate, the wells that were the most affected by evaporation. If there was cell growth at the start, evaporation would increase the cell density in the wells without requiring any cell growth. Other measurements that were likely affected by evaporation were the Anc-000 and SL-200-W4 curves in blank media (FIG. 11, top row). Both samples have wells with large abnormal peaks near the end of the run and both wells were plated on corners of the 96-well plate. The corner wells were observed to be the most affected by evaporation, causing the large OD₆₀₀ peaks. The other samples in blank LB also appear to be affected by evaporation, just not as much as the corner wells; small rises in OD₆₀₀ can be observed in the measurements of other samples in blank LB, such as for IL-000-W4 and SL-200-W4. All samples had one well on the outer row of the plate. Since all samples in blank LB appeared to be somewhat effect from evaporation, we filtered out the last 20 hours of measurements for all samples in blank LB.

When plated on agar, both the Ancestor and PL samples grew at least one colony on plates with 250 mM MgCl₂. It was therefore not surprising that these samples showed growth in 250 mM MgCl₂ during the adaptation analysis (FIG. 11, third row). However, these two samples had multiple isolates in the adaptation analysis (Anc-000, Anc-200, PL-000-Col1, PL-000-Col2, and PL-200) that were unable to grow in 250 mM MgCl₂. It was unexpected that the only two isolates that grew in 250 mM MgCl₂ were samples cultured in media without magnesium chloride.

PL-000-Col1 was a large, spreader colony and PL-000-Col2 was a smooth, round colony. PL-000-Col2 could not grow in any media with MgCl₂ whereas PL-000-Col1 was one of the best performing isolates. The OD₆₀₀ curves in blank LB for PL-000-Col1 and PL-000-Col2 are shaped differently than the other curves; most samples had smooth, “L” shaped curves while the PL samples had boxy, jagged measurements. WGS results identified these two PL samples as contaminated with Pseudomonas (Table 5). PL-000-Col1 had the lowest OD₆₀₀ measurements in blank LB. In 200 mM MgCl₂, most samples had “S” shaped curves whereas PL-000-Col1 had curves that were more vertical. This could have affected the downstream exponential growth rate and cell proliferation calculations by inflating the relative response of the sample in magnesium chloride; in 200 mM MgCl₂, PL-000-Col1 reached an average cell proliferation value of 130% the cell proliferation value in blank LB.

When averaged between triplicates, the OD₆₀₀ of the PL-000-Col1, PL-200, and SL-W4 reached a higher average in 200 mM MgCl₂ than in blank LB media. The highest OD₆₀₀ reached was one triplicate well for the PL-000-Col1 sample with a maximum OD₆₀₀ of 1.479 whereas the SL-W2 had the highest average OD₆₀₀ of 1.399. Both maximums were obtained in media with 200 mM MgCl₂. Unsurprisingly, the SL-W2 sample had the highest relative cell proliferation in 200 mM MgCl₂ of 140.8%. The PL-000-Col2 was the only sample that had a relative cell proliferation of less than 5% in 200 mM MgCl₂, considered as no growth. For the other samples in 200 mM MgCl₂, the cell proliferation values were 74.0, 94.0, 130.3, 129.3, 86.5, and 102.2 percent for Anc-000, Anc-200, PL-000-Col1, PL-200, IL-000, and SL-W4, respectively. The values for the average exponential growth rate in 200 mM MgCl₂ were 41.7, 32.0, 54.0, 26.5, 33.5, 22.2, and 42.3 percent for the Anc-000, Anc-200, PL-000-Col1, PL-200, IL-000, SL-W2, and SL-W4, respectively.

Substantial growth only occurred in blank media and 200 mM MgCl₂ thereby producing dose-response curves and ED50 values that are very similar, depending on only the growth obtained in 200 mM MgCl₂ to determine the outcome of the logistic fit. The exception being the PL-000-Col2 sample that could not grow in magnesium chloride, producing much lower ED50 values. The ED50 values represent the concentration of MgCl₂ at which the exponential growth rate or cell proliferation response in media without MgCl₂ is decreased by 50%, determined by a logarithmic fit of the data (Table 6).

TABLE 6

| | Relative Growth | Relative Cell |
|---|---|---|
| Sample Name | Rate ED50 | Proliferation ED50 |
| Anc-000 | 183.2 | 214.1 |
| Anc-200 | 189.0 | 215.8 |
| PL-000-Col1 | 203.0 | 240.2 |
| PL-000-Col2 | 86.7 | 128.1 |
| PL-200-W4 | 189.1 | 234.7 |
| IL-000-W4 | 184.1 | 214.1 |
| SL-200-W2 | 184.4 | 235.2 |
| SL-200-W4 | 194.0 | 234.9 |

Excluding PL-000-Col2, all exponential growth rate ED50 values fell between the Anc-000 value of 183.2 and that for PL-000-Col1 of 203.0, the highest value. The Anc-200 growth rate ED50 of 189.0 is slightly higher than that from the Anc-000 of 183.2, a similar trend seen for the cell proliferation where the Anc-200 is close but slightly higher than the Anc-000 with ED50 values of 215.8 and 214.1, respectively. For relative cell proliferation ED50 values, there is slightly more variation with samples ranging between 214.1 from the Anc-000 and IL-000-W4 and 240.2 for PL-000-Col1. PL-000-Col1 also has the highest ED50 for relative cell proliferation with an MgCl₂ concentration of 240.2. The relative cell proliferation ED50 values for the PL-200-W4, SL-200-W2, and SL-200-W4 are close behind with ED50 values of 234.7, 235.2, and 234.9, respectively. The IL-000 sample has an ED50 cell proliferation value that is farther from the other samples and closer to the ancestor samples with a value of 214.1 Lastly, like its growth rate ED50, the PL-000-Col2 sample had a very low ED50 value of 128.1.

### Example 12: Antibiotic Stress Adaptation Analysis

The two adaptation analyses for the antibiotic samples, described in Table 7, have similar trends across trials.

TABLE 7

| | | Time of | Cipro. in | Cipro. | |
|---|---|---|---|---|---|
| Name in | Sample | Sample | Culture Media | Concentration as | |
| Analysis | Origin | (weeks) | (ug/mL) | xMIC(0.06 ug/mL) | Breseg results |
| Anc-00 | Ancestor | — | 0.0 | 0x | Fully mapped |
| PL-00 | Permissive | 2 | 0.0 | 0x | Contaminated: Bacillus |
| | Layer | | | | |
| PL-0.6 | Permissive | 2 | 0.6 | 10x | Contaminated: Bacillus |
| | Layer | | | | |
| IL-LB | Interface | 2 | 0.0 | 0x | Contaminated: Bacillus |
| IL-15- | Interface | 2 | 15.0 | 250x | Contaminated: Bacillus |
| Col1 | | | | | |
| IL-15- | Interface | 2 | 15.0 | 250x | Contaminated: Bacillus |
| Col2 | | | | | |
| SL-15 | Stress Layer | 2 | 15.0 | 250x | Contaminated: Bacillus |
| BL-15 | Base | 2 | 0.6 then 15.0 | 10 then 250x | Contaminated: Bacillus |
| PL-15 | Permissive | 2 | 15.0 | 250x | Contaminated: Bacillus |
| | Layer | | | | |
| SL-00 | Stress Layer | 2 | 0.0 | 0x | Contaminated: Bacillus |
| BL-00 | Base | 2 | 0.0 | 0x | Contaminated: Bacillus |
| PL-C | Permissive | 2 | 0.0 | 0x | Contaminated: Bacillus |
| | Layer | | | | |
| | Community | | | | |
| IL-C | Interface | 2 | 0.0 | 0x | Contaminated: Bacillus |
| | Community | | | | |
| SL-C | Stress Layer | 2 | 0.0 | 0x | Contaminated: Bacillus |
| | Community | | | | |
| BL-C | Base | 2 | 0.0 | 0x | Contaminated: |
| | Community | | | | Paenibacillus |

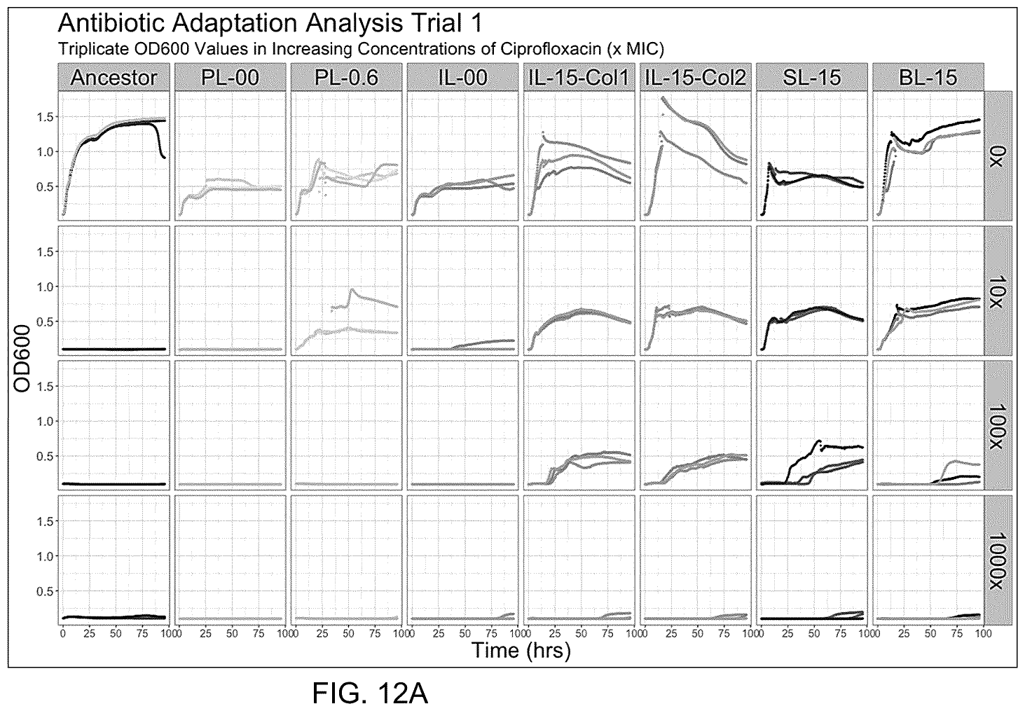

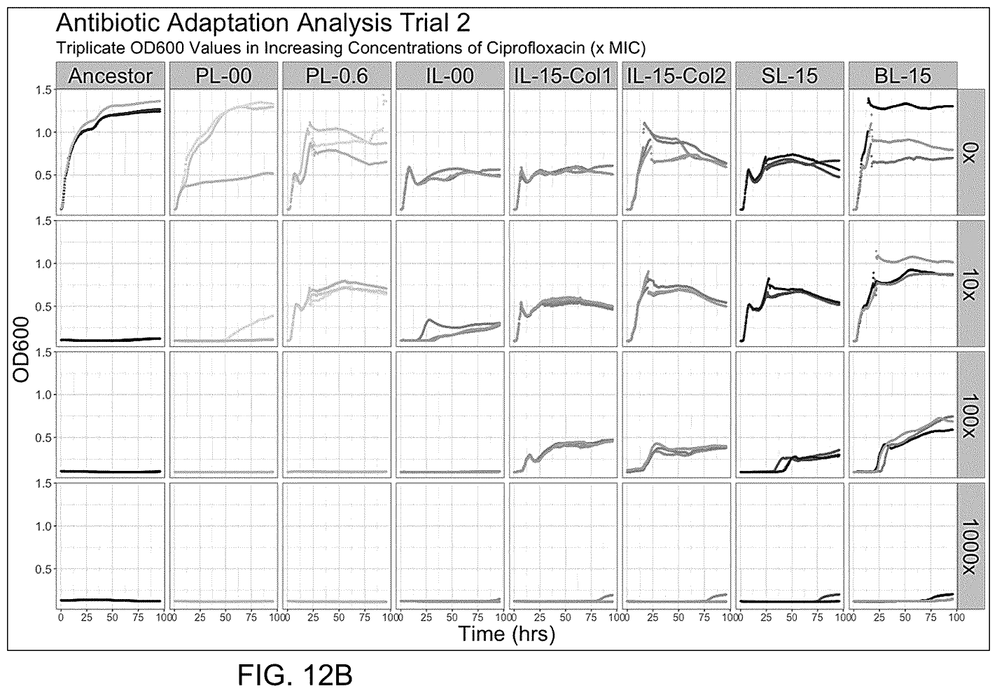

The top eight rows (above the dark line) correspond to samples used in the adaptation analysis shown in FIGS. 12A-12B. The lower seven rows correspond to samples that were sequenced but not used in the adaptation analysis. The name of the sample indicates the source of the sample, i.e., Ancestor, PL, IL, SL, or BL, and the concentration of ciprofloxacin that the sample was cultured in before analysis (0, 10, or 250×MIC) seen in FIG. 10. The BL-15 sample was grown on agar with 10×MIC then transferred to broth with 250×MIC. All samples were taken from MICALE at the end of the run, after two weeks. Samples named with a “C” for Community are broth cultures grown directly from the original MICALE sample, without prior isolation of a colony. Samples IL-15-Col1 and IL-15-Col2 Col2 indicate separate colonies with different morphologies from the same culture plate (FIG. 10b). The breseq results column describes if the sample was fully mapped to the E. coli reference genome or contaminated. No samples mapped to the reference genome.

In both trials, the observed growth response in ciprofloxacin increases from top to bottom in MICALE; the ancestor had no resistance to ciprofloxacin, the upper regions of MICALE, mainly PL, had mild resistance, and the samples from lower in MICALE, IL, SL, and BL, had the highest amount of resistance. In both trials, the IL-15-Col1, IL-15-Col2, SL-15, and BL-15 samples had OD₆₀₀ curves in ciprofloxacin up to 100×MIC. These same samples also each had at least one well with a small OD₆₀₀ measurement in 1000×MIC, starting at about 60 hours. The ancestor, on the other hand, did not show signs of growth in any media with ciprofloxacin in both trials. Lastly, the PL-00, PL-0.6, and IL-00 samples had mixed responses between the two trials but overall could not grow in 100 or 1,000×MIC.

All samples had similar-looking colony morphologies, regardless of the amount of ciprofloxacin in the agar media. The exception was the IL sample, which grew colonies with diverse morphology. This diversity, however, was only seen on agar with 250×MIC ciprofloxacin and not on the plates with lower amounts of ciprofloxacin.

During growth in the adaptation analysis, many of the samples formed visible clumps, possibly from biofilm or EPS production. This produced quick jumps in the OD₆₀₀ measurements that did not correspond to an increase in the cell count. Before further analysis, OD₆₀₀ measurements, therefore, were filtered out starting at the time of the first jump.

Table 8 lists the ED50 values derived from both Trial 1 and Trial 2 of the antibiotic adaptation analysis (FIGS. 12A-12B).

TABLE 8

| | | | Trial 2 | |
|---|---|---|---|---|
| | Trial 1 | | | Relative Cell |
| Sample | Relative Growth | Relative Cell | Relative Growth | Proliferation |
| Name | Rate ED50 | Proliferation ED50 | Rate ED50 | ED50 |
| Anc | n/a | n/a | n/a | n/a |
| PL-00 | a/a | n/a | 1.8786 | n/a |
| PL-0.6 | 8.6809 | 8.8578 | 15.5588 | 12.766 |
| IL-00 | n/a | n/a | 6.0863 | 9.2362 |
| IL-15- | 130.13 | 56.293 | 87.5896 | 186.32 |
| Col1 | | | | |
| IL-15- | 87.285 | 39.972 | 94.896 | 58.445 |
| Col2 | | | | |
| SL-15 | 48.657 | 119.389 | 54.9901 | 77.44 |
| BL-15 | 20.132 | 21.3079 | 172.053 | 111.977 |

| | MIC Relative | |
|---|---|---|
| | Growth Rate | MIC Relative Cell |
| | ED50 | Proliferation ED50 |
| Ancestor, | 0.23391 | 0.114513 |
| MIC | 0.240085 | 0.1583325 |
| | 0.203954 | 0.147608 |

Dose-response curves were fit to the exponential growth rate and cell proliferation values calculated from OD₆₀₀ curves for each sample. The ED50 value represents the concentration of ciprofloxacin, expressed as a factor of the MIC (0.06 μg/mL) at which a sample's response in media without antibiotic is decreased by 50%. The ED50 values for the ancestor were calculated using growth rates and cell proliferation values from OD₆₀₀ curves obtained during a test for the ancestor strain's MIC in ciprofloxacin. Dose-response curves were fit to each triplicate well grown during the MIC test to extract three ED50 values.

A similar pattern of increasing resistance from top to bottom in MICALE was observed in the average cell proliferation values. In trial 1, the Ancestor, PL-00, PL-0.6, IL-00, IL-15-Col1, IL-15-Col2, and BL-15 samples reached a cell proliferation value of less than 5% at 10, 10, 100, 100, 1000, 1000, and 1000×MIC, respectively. Therefore, these samples were considered to have no growth at those concentrations. The SL-15 sample in trial 1 grew in all concentrations, obtaining cell proliferation values over 5%. In 1000×MIC, the IL-00 had an average cell proliferation of 5.3% which could indicate growth. However, the average cell proliferation in 100×MIC was only 0.6% so the observed increase in cell proliferation in the higher antibiotic concentration is likely due to factors other than cell growth, such as evaporation.

While most of the lower-layer samples had cell proliferation values of less than 5% in 1000×MIC in trial 1, these samples were able to reach average cell proliferation values above 5% in trial 2. The IL-15-Col1, SL-15, and BL-15 from trial 2, therefore, are considered to have growth in all levels of ciprofloxacin tested. The other samples in trial 2, for the Anc, PL-00, PL-0.6, IL-00, and IL-15-Col2, reached average cell proliferation values lower than 5% in 10, 100, 100, 100, and 1000×MIC, respectively.

The highest overall exponential growth rate reached in trial 1 was 107.5% by IL-15-Col2 in 10×MIC. Despite this increase in growth rate, the cell proliferation value in 10×MIC for IL-15-Col2 was only 61.5%. IL-15-Col2 also had the fastest exponential growth rate in trial 2, reaching a growth rate in 10×MIC that is 101.6% the rate reached in blank LB. The cell proliferation value, however, was only 90.9% of the cell proliferation response seen in blank LB. In media with 100 and 1000×MIC ciprofloxacin, however, the IL-15-Col2 does not hold its spot as the sample with the highest growth rate. Instead, in 100×MIC, IL-15-Col1 and BL-15 had the highest relative growth rate of 54.4% in trial 1 and 66.6% in trial 2, respectively.

In trial 2, the four lower-region samples that showed growth in high levels of antibiotics, IL-15-Col1, IL-15-Col2, SL-15, and BL-15, all had very similar growth rates in blank media and 10×MIC ciprofloxacin. In 10×MIC, their growth rates were 90.5, 101.6, 90.6, and 97.2 percent of that in media without antibiotic, respectively. With exponential growth rates at over 90% their growth rate in blank media, these four samples are barely affected by ciprofloxacin at 10×MIC. Their cell proliferation values in 10×MIC were also very similar to that in blank LB.

The dose-response curves and ED50 values from the antibiotic analysis OD₆₀₀ curves were greatly affected by a sample's response in 10×MIC and 100×MIC. The dose-response curves were fit over a wide range of ciprofloxacin concentrations, 0 to 1000×MIC. Thus, there were some drastically different calculations for ED50 for samples that performed slightly differently in trial 1 versus trial 2 (Table 8). For example, the BL-15 sample performed much better in trial 2, especially in 100×MIC, resulting in an ED50 value of 172×MIC for the growth rate and 112×MIC for cell proliferation. In trial 1, however, the ED50 values for BL-15 were 20 for growth rate and 21 for cell proliferation. There are also differences in ED50 values between the growth rate and cell proliferation for one sample in the same trial. For example, in trial 2 the IL-15-Col1 sample had a growth rate ED50 value of 95 and a cell proliferation ED50 value of 186. This major difference is due to the sample's large decline in growth rate from 10 to 100×MIC compared to a smaller decrease in cell proliferation.

As with the adaptation analysis growth curves, the ED50 values exhibit clear grouping of the lower four samples, IL-15-Col1, IL-15-Col2, SL-15, and BL-15, with higher resistance to ciprofloxacin. The MICALE samples, which could not grow past 10×MIC, PL-00, PL-0.6, and IL-00, group lower than the four samples listed prior but still have large ED50 values compared to the Ancestor, which experienced no growth in any media with antibiotic.

Because the Ancestor could not grow in any ciprofloxacin, the data could not be properly fit to a logarithmic dose-response curve. Instead, we used the growth data from the MIC test to fit dose-response curves and obtain ED50 values. We wanted multiple ED50 values for the Ancestor to perform downstream statistical significance tests. The triplicate wells for the MIC test were split into three groups to fit three dose-response curves for three ED50 values. During the MIC test, the Ancestor was grown on a fine scale of ciprofloxacin, resulting in ED50 values less than 0.3×MIC, or 0.018 μg/mL ciprofloxacin. These ED50 values make sense because they are less than our observed MIC value of 0.06 μg/mL and the literature's value of 0.05 μg/mL.

### Example 13: Whole Genome Resequencing

Whole-genome resequencing and analysis with breseq performed on samples isolated from high salinity and chaotropic stress MICALE runs revealed samples with mutations distinct from the ancestor and reference genome. During analysis with breseq, both the ancestor and sample sequences were mapped to a reference genome for the E. coli strain. Genetic differences between the samples and reference genome were not counted if the ancestor sample had that same mutation.

Out of the seven samples sequenced from the high salinity run, only one showed distinct mutations. Of the six samples that did not have distinct mutations, four were unable to map, or only partially mapped, to the reference genome. The three remaining sequences from the PL-05, IL-05, and BL samples had high mapped percentages to the reference genome but only the IL sample had distinct mutations. Only the BL sample was used in the adaptation analysis, but the isolated culture used for sequencing does not come from the same colony that was isolated for the adaptation analysis.

The high salinity IL-05 sample sequence returned three mutations. Out of the eleven samples sequenced from the chaotropic stress run, three samples are predicted by breseq to have distinct mutations and two samples returned low mapped percentages. PL-000-Col1 and PL-000-Col2 were the only two samples with low mapped percentages. The three chaotropic-stress samples with distinct mutations are the SL-200-W2, SL-200-W4, and BL-200-W4. These two SL samples are the same as those used for the Adaptation Analysis. None of the antibiotic sample sequences, except for the ancestor strains, were able to map in breseq.

A key feature of the MICALE bioreactor is that it maintains an area of media permissive for wild type growth while providing access to areas with higher concentrations of the stressor. The regions of higher stress must increase to a level in which only adapted strains can survive. Between these permissive and inhospitable zones, there are intermediate levels of stress to facilitate adaptation. The above-described salinity measurements show that the upper region is low in salinity, permissive for wild type growth. Furthermore, the PL density measurements from the other runs show that the PL remained statistically similar to the TLM, media that is used to culture cells. The BL region density measurements show that the BL region is the same as the BLM. BLM media contains a concentration of stressor that is above the wild type strain's MIC; only adapted strains can grow well in these conditions. Lastly, the four individual sampling regions are statistically different in density (except for the BL and SL regions during the antibiotic run) showing how the density, and therefore the concentration of the stressor, increases from top to bottom in MICALE, providing intermediate steps for adaptation to occur.

The free mobility between media types for cells allowed by MICALE's non-structural, density-based design facilitates colonization of new areas with higher selective pressure but also limits the ability to distinguish between cells that are (a) alive and adapted, (b) alive but not adapted, or (c) dead. While the goal was to track the cell growth in each layer using optical density measurements, it was determined that optical density is more accurately a representation of the amount of biomass present. Un-adapted cells may not be able to grow in the stressful layer, but they will still float down from above and contribute to an increase in the OD₆₀₀ measurements in the lower layers. Indeed, observations lead to the conclusion that cells in the upper layer were forming aggregates that eventually settled downwards and collected in the BL layer. These observations support that the presence of cell matter in the lower layers does not mean the cells are stress-adapted strains that have colonized the BLM. Rather, other methods are needed to monitor the presence of live and adapted cells in the lower layers. For example, growing samples on spread plates and broth cultures with differing concentrations of the stressor, live/dead stains with flow cytometry, fluorescence microscopy, diluting samples into fresh media with stressor and monitoring optical density on a 96-well plate, and more.

In chemostats, turbidostats, and MICALE, a constant flow rate aids in maintaining a constant environment, which is important during ALE because it reduces variables such as low oxygen, spent nutrients, and low pH that could cause unintended stress. It is also important to control external factors during ALE to ensure that observed phenotypic change is a result of an intended adaptive pressure. If external pressures are not mediated and become stronger than the intended stressor, evolution could be driven towards undesired adaptations. Thus, it is important to ensure sterility of the system and media supply during experimental set up, sampling, and maintenance, e.g., filter replacement. As noted above, the MICALE apparatus used for initial testing could not be autoclaved, increasing the challenge to maintaining sterility. Accordingly, materials that can be autoclaved are preferred. Care should be taken when sampling from the valves to avoid release of media within the pump or pump tubing back into reactor chamber.

Limiting the growth rate of cells in the upper layer could be employed to help add evolutionary pressure to evolve stress resistance. Limiting a single nutrient in MICALE, rather than simply diluting media, could “starve” the cells in the upper layer and push evolution downwards. However, starvation may result in evolved cells with undesired phenotypes, such as increased ability to survive on the waste products of dead cells. Additionally, the amount or type of nutrient that is limited changes the level of persistence of the starved cells and could lead to different adaptations. To reduce the evolution of undesired phenotypes, the limited nutrient can be provided in excess in the BLM. Instead of adapting to the limited nutrient, such as by altering their metabolism, cells may instead focus on evolving to the stressor as they can sense an increase in the nutrient through chemotaxis. For example, carbohydrates could be limited in fresh media and glucose could be added to the BLM, in addition to sucrose.

Starving cells by limiting one nutrient in the TLM may also aid in the overall rate of adaptation by inducing the “general stress response” in E. coli. This response has been observed in response to nutritional stress, starvation, and environmental pressure such as osmotic pressure and low pH during which E. coli cells increase their rate of spontaneous mutation. During stress-induced mutagenesis, known as “adaptive mutation”, nondividing and starving cells can mutate and evolve their genome. Starving the cells, therefore, may serve as a “kick starter” for adaptation to the stressor.

An increasing gradient of stressor benefits the ALE experiment by facilitating adaptation to the stressor without imposing a time constraint on the cells. The density measurement results confirm that there is an area, i.e., a volume of media, permissive for wild type growth, an area with a high concentration of the stressor, and one or more area in between. Intermediate steps with lower concentrations of stress allow cells to gradually mutate to the stressor.

The longer MICALE is run, the greater the impact unintended stressors such as low oxygen, external cell matter, low nutrient levels, and decreased pH may have while the chosen stressor's impact reduces. The conditions in MICALE should be continuously monitored to determine whether unintended stressors have become too strong. For example, the amount of cellular biomass in the BLM pH in the lower region can indicate when too much cell matter has accumulated, and nutrients and oxygen have been depleted.

Observations of accumulated cell mass suggest that MICALE runs may preferably be limited to two weeks, without alteration of the BLM. Running MICALE for longer than two weeks may decrease the concentration of stressor and available resources in the system. Additionally, it has been noted that beneficial mutation rates decrease with longer evolutionary periods.

Nonetheless, the run length of MICALE can be easily increased by adding fresh BLM to the system after two weeks. Fresh BLM can be added through the bottom valve, replenishing nutrients while maintaining the density stratification. This can be taken further by increasing the concentration of the stressor in the fresh BLM to push the bounds of the adapted phenotypes, given that there is evidence of living cell populations in the lower region.

The Multilayered Instrument for Continuous Adaptive Laboratory Evolution (MICALE) disclosed herein provides a foundation for ALE experiments, employing density separation, and a resulting difference in the level of stress. The media in the upper region has low levels of stress thereby allowing growth of the wild type. Meanwhile, the density measurements of the SL and BL samples show that the lower regions have a concentration of stressor too high for wild type survival. This density separation is critical for creating a structured, heterogeneous environment with a region permissive for the wild type and a region with a level of stress that only adapted strains can survive in. The adaptation analysis provides phenotypic evidence that cells in the lower regions have higher levels of resistance than the ancestor. The flow rate controls the amount of biomass in the upper region but not the lower areas, maintaining a continuous culture of cells in the permissive upper region only. Altogether, the inventive MICALE allows for wild type survival in the upper region and selects for strains with increased resistance to stressors in the lower region.

1. A system for adaptive laboratory evolution of microorganisms, the system comprising:
a chemostat comprising a chamber, a fresh media inlet, and a waste media outlet, the chamber configured to retain a growth medium comprising density stratified layers, wherein an interface between the layers creates a gradient with an increasing concentration of a stressor and a nutrient progressing toward a bottom of the chamber, wherein an absence of physical barriers within the chamber permits the microorganisms to move freely within the growth medium; and
a plurality of access ports through the chamber, each access port disposed at a different height of the chemostat to provide access for extracting the growth medium at different vertical levels of the chamber.

2. The system of claim 1, wherein the density stratified layers comprise a top layer media (TLM) and a bottom layer media (BLM) each comprising the growth medium, wherein the TLM further comprises a dilutant added to the growth medium and wherein the nutrient within the growth medium increases a density of the BLM relative to the TLM.

3. The system of claim 2, wherein the growth medium is Luria broth, and wherein, in the TLM, the Luria broth is diluted to 50%.

4. The system of claim 1, wherein the nutrient is a carbohydrate,

5. The system of claim 4, wherein the carbohydrate is one or more sugar selected from the group consisting of sucrose, glucose, maltose, lactose, and trehalose.

6. The system of claim 1, wherein the microorganisms are Escherichia coli and the stressor compound comprises one or more of a saline compound, a chaotropic compound, and an antibiotic.

7. The system of claim 1, wherein each port of the plurality of access ports is configured for extracting the growth medium and associated micro-organisms located at different vertical levels within the chamber corresponding to a different layer within the growth medium.

8. The system of claim 7, wherein the different layers comprise a permissive layer (PL) configured for wild-type growth, an interface layer (IL) disposed below the PL, a stress layer (SL) below the IL, and a base layer (BL) disposed at the lower portion of the chamber.

9. The system of claim 8, wherein the fresh media inlet and the waste media outlet are configured to maintain a constant flow of the growth medium into and out of the chamber at a height of the chamber corresponding to the PL.

10. A method for adaptive laboratory evolution of microorganisms, the method comprising:
introducing microorganisms into a chemostat containing a growth medium comprising density stratified layers, wherein an interface between the layers creates a gradient with an increasing concentration of a stressor and a nutrient progressing toward a bottom of the chemostat, wherein an absence of physical barriers within the chemostat permits the microorganisms to move freely within the growth medium; and
extracting the growth medium via access ports disposed at different vertical levels of the chemostat.

11. The method of claim 10, wherein the density stratified layers comprise a top layer media (TLM) and a bottom layer media (BLM) each comprising the growth medium, wherein the TLM further comprises a dilutant added to the growth medium and wherein the nutrient within the growth medium increases a density of the BLM relative to the TLM.

12. The method of claim 11, wherein the growth medium is Luria broth, and wherein, in the TLM, the Luria broth is diluted to 50%.

13. The method of claim 10, wherein the nutrient is a carbohydrate,

14. The method of claim 13, wherein the carbohydrate is one or more sugar selected from the group consisting of sucrose, glucose, maltose, lactose, and trehalose.

15. The method of claim 10, wherein the microorganisms are Escherichia coli and the stressor compound comprises one or more of a saline compound, a chaotropic compound, and an antibiotic.

16. The method of claim 10, wherein each port of the plurality of access ports is configured for extracting the growth medium and associated micro-organisms located at different vertical levels within the chemostat corresponding to a different layer within the growth medium.

17. The method of claim 16, wherein the different layers comprise a permissive layer (PL) configured for wild-type growth, an interface layer (IL) disposed below the PL, a stress layer (SL) below the IL, and a base layer (BL) disposed at the lower portion of the chemostat.

18. The method of claim 17, wherein the fresh media inlet and the waste media outlet are configured to maintain a constant flow of the growth medium into and out of the chemostat at a height of the chemostat corresponding to the PL.
